SEQUENCE LISTING (3 sequences)

SEQ ID NO: 1 - protein, 24 aa, Artificial sequence
  Synthetic
CGGCGGCGGCGGCGGCGGCGGCGG

SEQ ID NO: 2 - protein, 5 aa, Artificial Sequence
  Synthetic
  MISC_FEATURE (4)..(4): Xaa may be any amino acid except for Proline
VPGXG

SEQ ID NO: 3 - protein, 18 aa, Artificial Sequence
  Synthetic
  REPEAT (6)..(12): Repeat unit repeating 160 times
  MISC_FEATURE (6)..(8): Xaa is V or G or A, which appears in a ratio of      VGA=178 in the repeating segment
  REPEAT (16)..(18): Repeat unit repeating 7 times
SKGPGXXXGVPGWPCGGC

(19) United States
(12) Patent Application Publication
(10) Pub. No.: US 2019/0083638 A1
(43) Pub. Date: Mar. 21, 2019

(54) NICLOSAMIDE-CONJUGATED POLYPEPTIDE NANOPARTICLES

(71) Applicant: Duke University, Durham, NC (US)

(72) Inventors: Wei Chen, Chapel Hill, NC (US); Jayanta Bhattacharyya, Durham, NC (US); Xiu-Rong Ren, Durham, NC (US); Robert A. Mook, Chapel Hill, NC (US); Jiangbo Wang, Durham, NC (US); Ivan Spasojevic, Durham, NC (US); Richard Premont, Durham, NC (US); Xinghai Li, Durham, NC (US); Ashutosh Chilkoti, Durham, NC (US)

(21) Appl. No.: 16/045,556

(22) Filed: Jul. 25, 2018

Related U.S. Application Data
(60) Provisional application No. 62/536,760, filed on Jul. 25, 2017.
(60) Provisional application No. 62/560,510, filed on Sep. 19, 2017.

(51) Int. Cl. A61K 47/64; A61K 31/167; A61P 35/00; A61P 19/02; A61P 3/08; A61K 47/69

(52) U.S. Cl. CPC A61K 47/64; A61K 31/167; A61K 47/6929; A61P 19/02; A61P 3/08; A61P 35/00

(57) ABSTRACT

Disclosed herein are conjugates of a therapeutic compound and polypeptides, such as a conjugate of niclosamide and an elastin-like polypeptide. These conjugates may form nanoparticles through self-assembly, which improve the solubility, bioavailability, and pharmacokinetic profiles of the therapeutic compound. Also disclosed are methods for treating cancer, parasite infection, bacterial infection, viral infection, metabolic diseases, Type II diabetes, NASH, NAFLD, artery constriction, endometriosis, neuropathic pain, rheumatoid arthritis, sclerodermatous graft-versus-host disease, and systemic sclerosis.

### CROSS-REFERENCE TO RELATED APPLICATION

This application claims the benefit of and priority to U.S. Provisional Application No. 62/536,760 filed on Jul. 25, 2017, and U.S. Provisional Application No. 62/560,510 filed on Sep. 19, 2017, the entire contents of all of which are incorporated herein by reference.

### STATEMENT REGARDING FEDERALLY SPONSORED RESEARCH

This invention was made with government support under grant numbers 5R01 CA172570 and 5K12-CA100639-08 awarded by the National Cancer Institute, R01 EB-00188 and R01 EB-007205 awarded by the National Institutes of Health, and BC123280 awarded by the Department of Defense. The government has certain rights in the invention.

### SEQUENCE LISTING

The sequence listing is filed with the application in electronic format only and is incorporated by reference herein. The sequence listing text file “028193-9271-US03_As_Filed_Sequence_Listing.txt” was created on Jul. 25, 2018, and is 1,296 bytes in size.

### FIELD

This disclosure relates to conjugates of a therapeutic compound and polypeptides, such as a conjugate of niclosamide and elastin-like polypeptides, and nanoparticles comprising such conjugates.

### BACKGROUND OF THE INVENTION

Wnt ligand binding to Frizzled/LRP receptors recruits Dishevelled to prevent the APC/Axin/GSK3β-mediated phosphorylation, ubiquitination and destruction of β-catenin, allowing β-catenin to accumulate and enter the nucleus to regulate specific gene activity. The Wnt signaling pathway plays a key role in tissue development and homeostasis, but is also dysregulated in many diseases. Specifically, in colorectal cancer (CRC), more than 80% of all sporadic and hereditary cancers exhibit hyperactivation of this pathway due to mutations in the adenomatous polyposis coli (APC) or β-catenin genes. Given the importance of Wnt signaling activity in promoting tumor formation and metastasis, therapies to target this pathway are medically needed. However, there is a lack of druggable Wnt signaling pathway drug targets downstream of APC and β-catenin, and because protein-protein interactions have traditionally been difficult to target with small drug-like molecules, drug discovery targeting this pathway at the level of these proteins has been problematic.

Niclosamide (NIC), a drug approved by the FDA for human use as an anthelmintic agent to treat tapeworm infections, has recently been found to promote Frizzled internalization. Subsequent studies show that NIC down-regulates Dishevelled and β-catenin, and inhibits colon cancer cell growth in vitro and in vivo. As a multi-functional drug, NIC has been found to inhibit the proliferation of tumor cell lines from multiple tumor types, e.g. breast, lung, prostate, lung, ovary, blood and pancreas, in addition to colon cancer, over an IC₅₀ range of 0.13-4 μM that overlaps with the IC₅₀ of inhibition of Wnt/β-catenin signaling. NIC also has anti-tumor activity in drug resistant cancers. NIC has been reported to inhibit key oncogenic signaling pathways in addition to Wnt, including Notch, mTOR, NF-kB, and STAT-3.

While the pharmacodynamic properties of NIC are appropriate for use in the gut lumen as an anthelmintic agent, its low solubility, low bioavailability and poor pharmacokinetic profile result in low plasma exposure when dosed orally. Efforts to improve its solubility have included the preparation of salt forms and of derivatives containing hydrophilic groups. In the search for STAT-3 inhibitors with improved solubility to treat cancer, water-solubilizing amine groups were incorporated into NIC. Recent efforts to identify nanoparticle formulations of NIC for use in cancer therapy have been reported, but these formulations did not result in significant improvement of pharmacological properties. In one study in which the pharmacokinetic parameters of a nanocrystal formulation of NIC was evaluated in vivo, the nanocrystal formulation did not significantly change the plasma concentration vs. time profile when administered intravenously (i.v.) to rats, though an increased tissue concentration at 2 hours was noted (Ye et al., Drug Dev. Ind. Pharm., 2014, 1-9).

Therefore, there is still a need for formulations of NIC with improved solubility, bioavailability, and pharmacokinetic profiles.

### SUMMARY

In one aspect, provided is a conjugate of formula (I), or a pharmaceutically acceptable salt thereof,

Z-(-L-D)ₚ (I)

wherein,

Z is a polypeptide having a cysteine-enriched segment;

p is 1 to 8;

each -L-D group is covalently attached to the cysteine-enriched segment;

L is linker;

D is

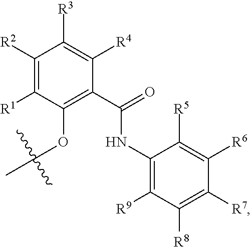

wherein
R¹, R², R³, R⁴, R⁵, R⁶, R⁸, and R⁹ at each occurrence are independently hydrogen, halogen, C₁-C₆ alkyl, C₁-C₆ haloalkyl, or C₁-C₆ alkoxy; and R⁷ at each occurrence is halogen, C₁-C₆ haloalkyl, —NO₂, or —SO₂—C₁-C₄ alkyl.

In another aspect, provided is a nanoparticle comprising the conjugate of formula (I) or a pharmaceutically acceptable salt thereof as described above. In certain embodiments, the nanoparticle as disclosed herein may be in the form of a micelle, in which the -L-D groups of the conjugate form a core of the nanoparticle.

In another aspect, provided is a pharmaceutical composition comprising the conjugate of formula (I) or a pharmaceutically acceptable salt thereof as described above and a pharmaceutically acceptable carrier.

In a further aspect, provided is a compound of formula (II), or a pharmaceutically acceptable salt thereof,

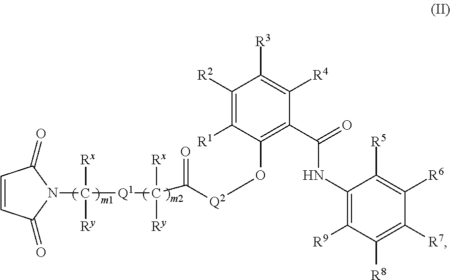

wherein,

Q¹ is bond, —O—, —NH—, aryl, cycloalkyl, heterocyclyl, heteroaryl, —(CH₂CH₂O)ₘ₃—, wherein the aryl, cycloalkyl, heterocyclyl, and heteroaryl are each optionally substituted by at least one C₁-C₄ alkyl, halogen, or C₁-C₄ haloalkyl;

Q² is bond, —O—CH(R^{w})—, -AAᵣ-, —Yₜ—, or -AAᵣ-Yₜ—, in which Y, if present, is attached to the

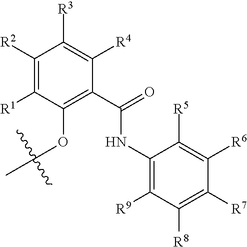

group;

R^{w} is H, C₁-C₆ alkyl, aryl, or cycloalkyl;

R^{x} and R^{y} at each occurrence are independently hydrogen or C₁-C₄ alkyl;

AA at each occurrence is independently an amino acid unit;

Y at each occurrence is independently a self-immolative spacer unit;

m1 is 0 to 10, provided that when Q¹ is bond, m1 is 1-10;

m2 is 0 to 10;

m3 is 0 to 20;

r is 1-10;

t is 1 or 2;

R¹, R², R³, R⁴, R⁵, R⁶, R⁸, and R⁹ at each occurrence are independently hydrogen, halogen, C₁-C₆ alkyl, C₁-C₆ haloalkyl, or C₁-C₆ alkoxy; and

R⁷ at each occurrence is halogen, C₁-C₆ haloalkyl, —NO₂, or —SO₂—C₁-C₄ alkyl.

In a further aspect, provided is a method of preparing a conjugate, comprising the steps of preparing a polypeptide having a cysteine-enriched segment; and reacting the polypeptide with a compound of formula (II) or a pharmaceutically acceptable salt thereof as described above to form the conjugate, wherein the compound is covalently attached to the cysteine-enriched segment of the polypeptide.

In a further aspect, provided is a method for treating disease in subjects in need thereof. Diseases to be treated by the compositions described herein may include, but are not limited to, cancer, parasite infection, bacterial infection, viral infection, metabolic diseases, Type II diabetes, NASH, NAFLD, artery constriction, endometriosis, neuropathic pain, rheumatoid arthritis, sclerodermatous graft-versus-host disease, and systemic sclerosis. The method for treating disease as disclosed herein may comprise administering to a subject in need thereof an effective amount of a pharmaceutical composition comprising the conjugate of formula (I) or a pharmaceutically acceptable salt thereof as described above and a pharmaceutically acceptable carrier. In one embodiment, the disease that may be treated by the method as disclosed herein is cancer.

The disclosure provides for other aspects and embodiments that will be apparent in light of the following detailed description and accompanying figures.

### BRIEF DESCRIPTION OF THE DRAWINGS

FIGS. 1A-1B show a representative structure of a chimeric polypeptide-niclosamide (CP—NIC) conjugate and a schematic illustration of the structure of a representative CP—NIC nanoparticle. The CP may be synthesized by genetically encoded synthesis in E. coli, and conjugated to NIC at the multiple Cys residues located at the carboxyl terminus (C-terminus) of the CP (FIG. 1A). Attachment of the hydrophobic drug NIC (triangles) to the CP may trigger self-assembly of the CP—NIC conjugate into a nanoparticle (for example, a cylindrical micelle) with a drug-rich core surrounded by hydrophilic polypeptides (for example, multiple long polypeptide chains surrounding the core) (FIG. 1B).

FIGS. 2A-2I show the results of the characterization of representative CP—NIC nanoparticles. FIG. 2A shows MALDI-MS of CP and CP—NIC conjugate. FIG. 2B shows DLS measurement of CP—NIC conjugate (n=3). FIG. 2C shows angular dependence of Rh of CP—NIC nanoparticles. FIG. 2D shows Partial Zimm plot of Kc/R vs q2 of CP—NIC conjugate. FIG. 2E shows cryo-TEM image of CP—NIC nanoparticles (Scale 200 nm). FIG. 2F shows AFM images of CP—NIC conjugate. FIG. 2G shows transition temperature (Tt). FIG. 2H shows critical aggregation concentration (CAC) of CP—NIC conjugate. FIG. 2I shows SDS-PAGE of CP and CP—NIC conjugate.

FIGS. 3A-3B show the in vitro activity of representative CP—NIC nanoparticles. FIG. 3A shows cell viability in the presence of the indicated doses of free NIC, CP—NIC (equivalent dose to free NIC), or unconjugated CP (equivalent to CP—NIC) in HCT-116 cells (n=3, mean+95% Cl). FIG. 3B shows inhibition of Wnt/β-catenin signaling measured as cytosolic β-catenin level, as well as Wnt-targets c-myc and cyclin D1, by the indicated doses of CP—NIC versus free NIC in HCT-116 cells. Actin is a loading control.

FIG. 4 shows plasma pharmacokinetics of a representative CP—NIC nanoparticles. CD1 mice were dosed with CP—NIC (i.v., 128 mg/kg). Blood samples were obtained 0.5 h prior to dosing and at 0.08, 0.17, 0.33, 0.67, 1.5, 4, 8, 12, 24 hours after drug administration (n=4 per time point). Quantification of NIC in mouse plasma was done by LC/MS-MS and reported as ng/ml. A non-compartment model was fitted to the plasma NIC concentration, which yielded a terminal half-life of 4.2 h for CP—NIC (mean+95% Cl, n=4). The dotted line denotes the IC₅₀ of NIC inhibition of Wnt/β-catenin signaling in the Wnt-stimulated TOPFlash assay.

FIGS. 5A-5B show in vivo anti-tumor activity of a representative CP—NIC nanoparticles. For FIG. 5A and FIG. 5B, tumor cells (HCT-116) were implanted in the right flank of male nude mice on day zero. When the tumor volume reached ˜100 mm3, mice were treated intravenously every third day for two weeks with PBS (n=8), unconjugated NIC (5 mg/kg BW, n=8) or CP—NIC (20 mg NIC equiv/kg BW, n=8). FIG. 5A shows tumor volume up to day 30 (mean±95% Cl, n=8). p>0.0001 for CP—NIC, NIC or PBS treatment. At day 10, comparison was made among groups using the Tukey test. FIG. 5B shows cumulative survival of mice (Kaplan-Meier).

FIG. 6 shows DLS of representative CP—NIC conjugate. Dynamic light scattering was used to measure particle radius at 25° C. and at 10 μM concentration in PBS after filtration through an Anotop™ syringe filter with 0.22 μm size pores (Whatman; Florham Park, N.J.) using a DynaPro™ Plate Reader (Wyatt Technology; Santa Barbara, Calif.). The histograms (n=3) were obtained after regularization fits to determine the hydrodynamic radius as weighted by the percent by mass.

FIGS. 7A-7B show the Cryo-TEM micrograph of a representative CP—NIC conjugate.

FIG. 8 shows the AFM images of a representative CP.

FIG. 9 shows the determination of transition temperature (Tt) of a representative CP at concentration ranging from 5-50 μM in PBS.

FIG. 10 shows the determination of the hydrodynamic radius of NIC from a representative CP—NIC at pH7.4 and 6.5 and at 25° C.

FIGS. 11A-11B show the change in body weight of mice. FIG. 11A shows the dose escalation in mice bearing subcutaneous HCT116 tumor. Solutions were administered starting at day 0. Mice were treated intravenously q3d for two weeks with representative CP—NIC at 5-20 mg NIC equiv/kg BW. Points represent the mean±SD (n=4). FIG. 11B shows the body weight of mice (up to 16 days) bearing subcutaneous HCT116 tumor and treated with CP—NIC (20 mg NIC equiv/kg BW, n=8), unconjugated NIC (5 mg/kg BW, n=8), and PBS (n=8) as mentioned in FIG. 5A and FIG. 5B for two weeks.

FIG. 12 shows the in vivo anti-tumor activity of representative CP—NIC nanoparticles. % change in the tumor volume up to day 30 (mean±95% Cl, n=8) with mice bearing subcutaneous HCT116 tumor were treated intravenously every third day for two weeks with PBS (n=8), unconjugated NIC (5 mg/kg BW, n=8) or CP—NIC (20 mg NIC equiv/kg BW, n=8) as mentioned in FIG. 5A and FIG. 5B.

### DETAILED DESCRIPTION

The present disclosure generally relates to polypeptide-drug conjugates. The polypeptides include, for example, recombinant chimeric polypeptides (CPs). The drug compounds include, for example, niclosamide (NIC) or similar compounds. In one particular aspect, disclosed here are conjugates of chimeric polypeptides and niclosamide (CP—NIC) or similar drug compounds, which may self-assemble into nanoparticles. The nanoparticles may exhibit Wnt signaling inhibition similar to that of free niclosamide in colon cancer cells. Remarkably, the CP—NIC nanoparticles disclosed herein may be used in a pharmaceutical composition to increase the plasma exposure of niclosamide as compared to administration of free niclosamide, and enhance the efficacy of the drug in reducing tumor growth of human colon cancer. The niclosamide-loaded nanoparticles disclosed herein may increase plasma exposure to niclosamide, extend its duration of exposure and improve its in vivo efficacy, thereby overcoming the barriers to the clinical translation of niclosamide to treat cancer. The conjugates disclosed herein enable the study of niclosamide in vivo in other diseases for which niclosamide has demonstrated biological activity. Thus, the conjugates disclosed herein may provide a breakthrough to treat diseases ranging from cancer, viral infection, bacterial infection and metabolic diseases in which Wnt signaling is implicated. The compositions disclosed herein may be used to treat cancer, parasite infection, bacterial infection, viral infection, metabolic diseases, Type II diabetes, NASH, NAFLD, artery constriction, endometriosis, neuropathic pain, rheumatoid arthritis, sclerodermatous graft-versus-host disease, and systemic sclerosis. In some embodiments, the attachment of multiple copies of a drug to the carboxyl terminus of the CP may trigger self-assembly of the CP-drug conjugate into spherical nanoparticles. CP nanoparticles may improve the delivery of chemotherapeutics. CP nanoparticles may improve the delivery of hydrophobic chemotherapeutics that are clinically approved. CP nanoparticles may be used to improve the delivery of drug candidates that have been discarded in drug development pipelines due to physicochemical properties that made delivery challenging. CP nanoparticles may be used to improve the delivery of drug candidates that have been discarded in drug development pipelines due to low water solubility and poor bioavailability.

CP nanoparticles incorporating drugs may result in increased effectiveness compared to the drug alone. CP nanoparticles incorporating chemotherapeutics may lead to significantly better tumor regression than the drug alone. CP-niclosamide nanoparticles may lead to significantly better tumor regression than niclosamide alone.

The conjugation of multiple copies of niclosamide to CP may trigger self-assembly of the conjugate into nanoparticles. The conjugation of multiple copies of niclosamide to CP may trigger self-assembly of the conjugate into nanoparticles in aqueous solution. The CP-niclosamide nanoparticles may exhibit Wnt signaling inhibition similar to that of free niclosamide in colon cancer cells. CP-niclosamide nanoparticles may provide a valuable preclinical research tool to study the effectiveness of NIC in preclinical models of cancer and other diseases known to be affected by NIC. CP-niclosamide nanoparticles may be better tolerated by a subject than free niclosamide. When injected intravenously, CP-niclosamide nanoparticles may be better tolerated than free niclosamide. A higher dose of CP-niclosamide nanoparticles than free niclosamide may be tolerated by the subject. A higher dose of CP-niclosamide nanoparticles may lead to increased plasma concentration as a function of time and to longer duration of exposure, compared to free niclosamide. CP-niclosamide may increase the tissue exposure of niclosamide when dosed intravenously. CP-niclosamide may exhibit a greater anti-cancer activity than free niclosamide in a colon cancer xenograft model. CP-niclosamide may exhibit a greater anti-cancer activity than free niclosamide in a colon cancer xenograft model, with no observable adverse effects over two weeks of intravenous dosing. CP-niclosamide may be administered in subjects who would benefit from niclosamide treatment. CP-niclosamide may be administered in subjects to treat cancer, parasite infection, bacterial infection, viral infection, metabolic diseases, Type II diabetes, NASH, NAFLD, artery constriction, endometriosis, neuropathic pain, rheumatoid arthritis, sclerodermatous graft-versus-host disease, and systemic sclerosis. CP-niclosamide may be administered in subjects with cancer. CP-niclosamide may be administered in subjects with colon cancer.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated. All possible combinations of numerical values between and including the lowest value and the highest value enumerated are to be considered to expressly stated in this application.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

“Administration” or “administering” refers to delivery of an agent, such a compound or a conjugate as disclosed herein, by any appropriate route to achieve the desired effect. Suitable administration routes may include, but are not limited to, oral, sublingual, intramuscular, subcutaneous, intravenous, transdermal, topical, parenteral, buccal, rectal, and via injection, inhalation, and implants. In some embodiments, the conjugate may be administered via parenteral routes, for example, intradermal, intramuscular or subcutaneous administration. In some embodiments, the conjugate is administered intravenously, intra-arterially, or intraperitoneally to the subject.

As used herein, the term “alkyl” refers to a linear or branched hydrocarbon radical, preferably having 1 to 30 carbon atoms, 1 to 10 carbon atoms, 1 to 6 carbon atoms, or 1 to 4 carbon atoms. The term “C₁-C₄ alkyl” is defined to include alkyl groups having 1, 2, 3, or 4 carbons in a linear or branched arrangement. For example, “C₁-C₄ alkyl” specifically includes methyl, ethyl, n-propyl, i-propyl, n-butyl, t-butyl, and i-butyl. The term “C₁-C₆ alkyl” is defined to include alkyl groups having 1, 2, 3, 4, 5 or 6 carbons in a linear or branched arrangement. For example, “C₁-C₆ alkyl” specifically includes methyl, ethyl, n-propyl, i-propyl, n-butyl, t-butyl, i-butyl, pentyl and hexyl.

The term “alkoxy” as used herein, refers to an alkyl group, as defined herein, appended to the parent molecular moiety through an oxygen atom. Representative examples of alkoxy include, but are not limited to, methoxy, ethoxy, propoxy, 2-propoxy, butoxy and tert-butoxy.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

The term “aryl” as used herein, refers to a phenyl group, or bicyclic aryl or tricyclic aryl fused ring systems. Bicyclic fused ring systems are exemplified by a phenyl group appended to the parent molecular moiety and fused to a phenyl group. Tricyclic fused ring systems are exemplified by a phenyl group appended to the parent molecular moiety and fused to two other phenyl groups. Representative examples of bicyclic aryls include, but are not limited to, naphthyl. Representative examples of tricyclic aryls include, but are not limited to, anthracenyl. The monocyclic, bicyclic, and tricyclic aryls are connected to the parent molecular moiety through any carbon atom contained within the rings.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifying a patient having CRC. A description of ROC analysis is provided in P. J. Heagerty et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25th-75th percentile range, preferably a value that corresponds to the 25th percentile, the 50th percentile or the 75th percentile, and more preferably the 75th percentile. Such statistical analyses may be performed using any method known in the art and can be implemented through any number of commercially available software packages (e.g., from Analyse-it Software Ltd., Leeds, UK; StataCorp LP, College Station, Tex.; SAS Institute Inc., Cary, N.C.). The healthy or normal levels or ranges for a target or for a protein activity may be defined in accordance with standard practice. A control may be a subject, or a sample therefrom, whose disease state is known. The subject, or sample therefrom, may be healthy, diseased, diseased prior to treatment, diseased during treatment, or diseased after treatment, or a combination thereof.

The term “cycloalkyl” as used herein, refers to a carbocyclic ring system containing three to ten carbon atoms, zero heteroatoms and zero double bonds. Representative examples of cycloalkyl include, but are not limited to, cyclopropyl, cyclobutyl, cyclopentyl, cyclohexyl, cycloheptyl, cyclooctyl, cyclononyl, and cyclodecyl.

“Effective amount” refers to a dosage of the compounds or compositions effective for eliciting a desired effect. This term as used herein may also refer to an amount effective at bringing about a desired in vivo effect in an animal, preferably, a human, such as treatment of a disease.

The term “expression vector” indicates a plasmid, a virus or another medium, known in the art, into which a nucleic acid sequence for encoding a desired protein can be inserted or introduced.

The term “halogen” or “halo” as used herein, means Cl, Br, I, or F.

The term “haloalkyl” as used herein, means an alkyl group, as defined herein, in which one, two, three, four, five, six, seven or eight hydrogen atoms are replaced by a halogen. The haloalkyl may be a C₁-C₆ haloalkyl or C₁-C₄ haloalkyl. Example of haloalkyl includes chloromethyl, fluoromethyl, and trifluoromethyl.

As used herein, the term “heteroaryl” refers to a monocyclic heteroaryl or a bicyclic heteroaryl. The monocyclic heteroaryl is a five- or six-membered ring. The five-membered ring contains two double bonds. The five-membered ring may contain one heteroatom selected from O or S; or one, two, three, or four nitrogen atoms and optionally one oxygen or sulfur atom. The six-membered ring contains three double bonds and one, two, three or four nitrogen atoms. Representative examples of monocyclic heteroaryl include, but are not limited to, furanyl, imidazolyl, isoxazolyl, isothiazolyl, oxadiazolyl, 1,3-oxazolyl, pyridinyl, pyridazinyl, pyrimidinyl, pyrazinyl, pyrazolyl, pyrrolyl, tetrazolyl, thiadiazolyl, 1,3-thiazolyl, thienyl, triazolyl, and triazinyl. The bicyclic heteroaryl includes a monocyclic heteroaryl fused to a phenyl, or a monocyclic heteroaryl fused to a monocyclic cycloalkyl, or a monocyclic heteroaryl fused to a monocyclic cycloalkenyl, or a monocyclic heteroaryl fused to a monocyclic heteroaryl, or a monocyclic heteroaryl fused to a monocyclic heterocycle. Representative examples of bicyclic heteroaryl groups include, but are not limited to, benzofuranyl, benzothienyl, benzoxazolyl, benzimidazolyl, benzoxadiazolyl, 6,7-dihydro-1,3-benzothiazolyl, imidazo[1,2-a]pyridinyl, indazolyl, indolyl, isoindolyl, isoquinolinyl, naphthyridinyl, pyridoimidazolyl, quinazolinyl, quinolinyl, thiazolo[5,4-b]pyridin-2-yl, thiazolo[5,4-d]pyrimidin-2-yl, and 5,6,7,8-tetrahydroquinolin-5-yl.

As used herein, the term “heterocycle” or “heterocyclyl” refers to a monocyclic heterocycle, a bicyclic heterocycle, or a tricyclic heterocycle. The monocyclic heterocycle is a three-, four-, five-, six-, seven-, or eight-membered ring containing at least one heteroatom independently selected from the group consisting of oxygen, nitrogen, phosphorus and sulfur. The three- or four-membered ring contains zero or one double bond, and one heteroatom selected from the group consisting of oxygen, nitrogen, phosphorus and sulfur. The five-membered ring contains zero or one double bond and one, two or three heteroatoms selected from the group consisting of oxygen, nitrogen, phosphorus and sulfur. The six-membered ring contains zero, one or two double bonds and one, two, or three heteroatoms selected from the group consisting of oxygen, nitrogen, phosphorus and sulfur. The seven- and eight-membered rings contains zero, one, two, or three double bonds and one, two, or three heteroatoms selected from the group consisting of oxygen, nitrogen, phosphorus and sulfur. Representative examples of monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, phosphinane, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydropyrimidinyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, trithianyl, and 2,5-dioxo-pyrrolidinyl. The bicyclic heterocycle is a monocyclic heterocycle fused to a phenyl group, or a monocyclic heterocycle fused to a monocyclic cycloalkyl, or a monocyclic heterocycle fused to a monocyclic cycloalkenyl, or a monocyclic heterocycle fused to a monocyclic heterocycle, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. Representative examples of bicyclic heterocycles include, but are not limited to, benzopyranyl, benzothiopyranyl, chromanyl, 2,3-dihydrobenzofuranyl, 2,3-dihydrobenzothienyl, azabicyclo[2.2.1]heptyl (including 2-azabicyclo[2.2.1]hept-2-yl), 2,3-dihydro-1H-indolyl, isoindolinyl, octahydrocyclopenta[c]pyrrolyl, octahydropyrrolopyridinyl, 9-phosphabicyclo[3.3.1]nonane, 8-phosphabicyclo[3.2.1]octane, and tetrahydroisoquinolinyl. Tricyclic heterocycles are exemplified by a bicyclic heterocycle fused to a phenyl group, or a bicyclic heterocycle fused to a monocyclic cycloalkyl, or a bicyclic heterocycle fused to a monocyclic cycloalkenyl, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan, hexahydro-1H-1,4-methanocyclopenta[c]furan, aza-admantane (1-azatricyclo[3.3.1.1^{3,7}]decane), oxa-adamantane (2-oxatricyclo[3.3.1.1^{3,7}]decane), and 2,4,6-trioxa-8-phosphatricyclo[3.3.1.13,7]decane. Heterocyclic groups of the present invention may contain one or more oxo groups (═O) or thioxo (═S) groups attached to the ring.

The term “host cell” is a cell that is susceptible to transformation, transfection, transduction, conjugation, and the like with a nucleic acid construct or expression vector. Host cells can be derived from plants, bacteria, yeast, fungi, insects, animals, etc. In some embodiments, the host cell includes Escherichia coli.

“Polynucleotide” as used herein can be single stranded or double stranded, or can contain portions of both double stranded and single stranded sequence. The polynucleotide can be nucleic acid, natural or synthetic, DNA, genomic DNA, cDNA, RNA, or a hybrid, where the polynucleotide can contain combinations of deoxyribo- and ribo-nucleotides, and combinations of bases including uracil, adenine, thymine, cytosine, guanine, inosine, xanthine hypoxanthine, isocytosine, and isoguanine. Polynucleotides can be obtained by chemical synthesis methods or by recombinant methods.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, e.g., enzymatic domains, extracellular domains, transmembrane domains, pore domains, and cytoplasmic tail domains. Domains are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three dimensional structure of a polypeptide monomer. “Quaternary structure” refers to the three dimensional structure formed by the noncovalent association of independent tertiary units. A “motif” is a portion of a polypeptide sequence and includes at least two amino acids. A motif may be 2 to 20, 2 to 15, or 2 to 10 amino acids in length. In some embodiments, a motif includes 3, 4, 5, 6, or 7 sequential amino acids. The polypeptide has a C-terminus, which is a carboxy-terminus (e.g. —COOH) at one end of the polypeptide sequence.

“Recombinant” when used with reference, e.g., to a cell, or nucleic acid, protein, or vector, indicates that the cell, nucleic acid, protein, or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed, or not expressed at all.

“Sample” or “test sample” as used herein can mean any sample in which the presence and/or level of a polypeptide, conjugate, or target is to be detected or determined. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Subject” as used herein can mean a mammal that wants or is in need of the herein described conjugates. The subject may be a human or a non-human animal. The subject may be a mammal. The mammal may be a primate or a non-primate. The mammal can be a primate such as a human; a non-primate such as, for example, dog, cat, horse, cow, pig, mouse, rat, camel, llama, goat, rabbit, sheep, hamster, and guinea pig; or non-human primate such as, for example, monkey, chimpanzee, gorilla, orangutan, and gibbon. The subject may be of any age or stage of development, such as, for example, an adult, an adolescent, or an infant.

The term “treatment,” as used herein in the context of treating a condition, pertains generally to treatment and therapy, whether of a human or an animal (e.g. in veterinary applications), in which a desired therapeutic effect is achieved. For example, treatment includes prophylaxis and can ameliorate or remedy the condition, disease, or symptom, or treatment can inhibit the progress of the condition or disease (e.g., reduce the rate of disease/symptom progression or halt the rate of disease/symptom progression).

“Zwitterionic” or “zwitterion” refers to a molecule with net charge of zero, but including negative and positive charges on independent individual atoms within the molecule. The charged atoms are joined by one or more covalent bonds. A polypeptide may be zwitterionic.

If substituents are described as being “independently selected” from a group, each substituent is selected independent of the other. Each substituent, therefore, may be identical to or different from the other substituent(s).

### 2. Conjugate

Provided herein are conjugates of formula (I), or a pharmaceutically acceptable salt thereof,

Z-(-L-D)ₚ (I)

wherein,

Z is a polypeptide having a cysteine-enriched segment;

p is 1 to 8;

each -L-D group is covalently attached to the cysteine-enriched segment;

L is linker;

D is

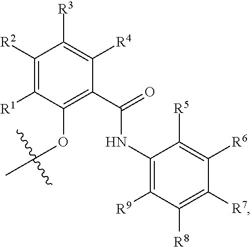

wherein
R¹, R², R³, R⁴, R⁵, R⁶, R⁸, and R⁹ at each occurrence are independently hydrogen, halogen, C₁-C₆ alkyl, C₁-C₆ haloalkyl, or C₁-C₆ alkoxy; and
R⁷ at each occurrence is halogen, C₁-C₆ haloalkyl, —NO₂, or —SO₂—C₁-C₄ alkyl.

In certain embodiments, the conjugate of formula (I) contains one or more -L-D groups attached to the polypetide via covalent bonds formed between each L group and a functional group on the peptide. Suitable functional group on the polypeptide include, but are not limited to, sulfhydryl (—SH), amino, hydroxyl, or carboxyl groups. In some embodiments, the functional group on the polypeptide to which L is attached is the sulfhydryl group of a cysteine residue. In a particular embodiment, the one or more -L-D groups of formula (I) are attached to the cysteine-enriched segment of the polypeptide via covalent bonds between the L moieties and the —SH groups in the cysteine residues of the cysteine-enriched segment.

2.1 Polypeptide

The conjugate of the present disclosure may include a polypeptide. In some embodiments, the polypeptide is a genetically encoded elastin-based chimeric polypeptide (CP). In some embodiments, the CP comprises an elastin-like polypeptide (ELP) fused to a short (Cys-Gly-Gly)₈ (SEQ ID. NO:1) peptide segment that provides thiol sites for chemical conjugation of chemotherapeutic drugs. CPs may be thermally responsive. CPs may display lower critical solution temperature (LCST) phase transition behavior. The protein may go from a soluble state to an insoluble coacervate phase upon raising the solution temperature above its cloud point, which may be referred to as the inverse transition temperature.

2.1.1 Elastin-Like Polypeptides

Elastin-like polypeptides (ELPs) may be biopolymers derived from human elastin. ELPs may have a lower critical solution temperature phase transition behavior. The lower critical solution temperature phase transition behavior of ELPs and biocompatibility may make ELPs useful materials for stimulus-responsive applications in biological environments. ELPs may be used for drug delivery. ELPs may be used to deliver biologic therapeutics, radionuclides, and small molecule drugs. ELPs may be used to deliver a variety of anatomical sites for the treatment of diseases. ELPs may be used to deliver biologic therapeutics, radionuclides, and small molecule drugs to treat diseases, including, but not limited to cancer, type 2 diabetes, osteoarthritis, and neuroinflammation.

The ELP may be a biopolymer such as, for example, a polypeptide. The ELP biopolymer may comprise a Val-Pro-Gly-Xaa-Gly (VPGXG, SEQ ID. NO:2) pentapeptide repeat. The Xaa may be a guest residue. The Xaa guest residue may be any amino acid except Pro. For example, the ELP may comprise the pentapeptide repeat sequence (VPGXG)ₙ, wherein X may be any amino acid except for proline, and n may be an integer greater than or equal to 1. In some embodiments, n is 10, 20, 30, 40, 50, 60, 70, 80, 90, 100, 110, 120, 130, 140, 150, 160, 170, 180, 190, 200, 210, 220, 230, 240, 250, 260, 270, 280, 290, or 300. In some embodiments, n may be less than 500, less than 400, less than 300, less than 200, or less than 100. In some embodiments, n may be between 1 and 500, between 1 and 400, between 1 and 300, or between 1 and 200. In some embodiments, n is 60, 120, or 180. In some embodiments n is 160.

In some embodiments, niclosamide may be covalently attached to a CP. The CP may comprise a cysteine-enriched segment or sequence (“segment” and “sequence” are used interchangeably herein when referencing the cysteine-enriched portion(s)). For example, the cysteine-enriched sequence may be GGC, which may be repeated 2 or more times. The CP used for conjugation to niclosamide may comprise the sequence SKGPG-(XGVPG)₁₆₀-WPC(GGC)₇ (single amino acid codes), where the guest residue X═V:G:A in a 1:7:8 ratio (SEQ ID NO:3). The cysteine-enriched sequence may be at or near the C-terminus of the polypeptide. The cysteine-enriched sequence may be at or near the C-terminus of the ELP. The polypeptide may be a CP.

2.2 Linker

The L group of formula (I) is a linker unit that links the polypeptide and the drug molecule of the conjugate disclosed herein. In general, the linker unit contains a functional group that forms a bond with a functional group on the polypeptide. Suitable functional group on the polypeptide include, but are not limited to, sulfhydryl (—SH), amino, hydroxyl, or carboxyl groups. In some embodiments, the linker unit contains a functional group capable of forming a covalent bond with the —SH group on the polypeptide, such as the —SH groups of the cysteine residues in the cysteine-enriched segment as disclosed herein. Useful functional groups that are reactive toward —SH include, for example maleimide, haloacetyl (bromo- or iodo-), and pyridyldisulfide.

In some embodiments, L is attached to the cysteine residue of the polypeptide through a maleimide functional group. In some embodiments, L has the formula (L-1):

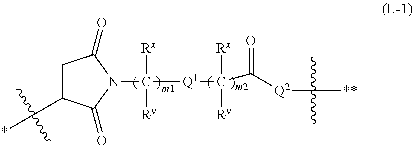

wherein

Q¹ is bond, —O—, —NH—, aryl, cycloalkyl, heterocyclyl, heteroaryl, —(CH₂CH₂O)ₘ₃—, wherein the aryl, cycloalkyl, heterocyclyl, and heteroaryl are each optionally substituted by at least one C₁-C₄ alkyl, halogen, or C₁-C₄ haloalkyl;

Q² is bond, —O—CH(R^{w})—, -AAᵣ-, —Yₜ—, or -AAᵣ-Yₜ—, in which Y, if present, is attached to D;

R^{w} is H, C₁-C₆ alkyl, aryl, or cycloalkyl;

R^{x} and R^{y} at each occurrence are independently hydrogen or C₁-C₄ alkyl;

AA at each occurrence is independently an amino acid unit;

Y at each occurrence is independently a self-immolative spacer unit;

m1 is 0 to 10, provided that when Q1 is bond, m1 is 1-10;

m2 is 0 to 10;

m3 is 0 to 20;

r is 1-10;

t is 1 or 2;

* indicates attachment to the polypeptide Z of formula (I);

** indicates attachment to the D group of formula (I).

In some embodiments, Q² is bond and L is of formula (L-2)

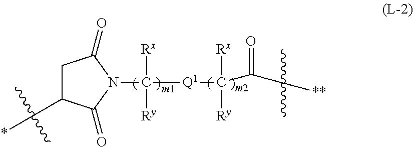

wherein Q¹, R^{x}, R^{y}, m1, and m2 are as defined in formula (L-1).

In some embodiments, Q² is bond and Q¹ is bond. In some embodiments, Q² is bond, Q¹ is bond, m2 is 0, and L is of formula (L-2a)

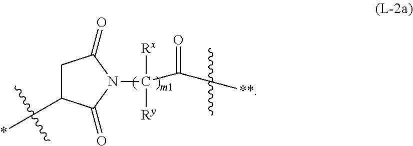

In some embodiments, L is of formula (L-2b)

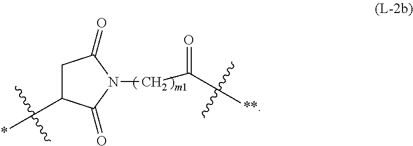

In a particular embodiment, L is of formula (L-2c)

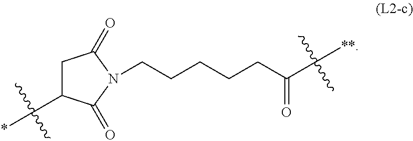

In some embodiments, Q¹ is not bond. In some embodiments, Q¹ is an optionally substituted cycloalkyl, aryl, or —(CH₂CH₂O)ₘ₃—.

In some embodiments, Q¹ is a cycloalkyl, such as cyclopropyl, cyclobutyl, cyclopentyl, cyclohexyl, cycloheptyl, cyclooctyl, cyclononyl and cyclodecyl, each of which may be optionally substituted. In some embodiments, Q¹ is an optionally cyclohexyl and L is of formula (L-3)

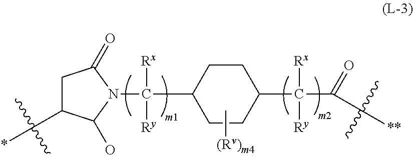

wherein R^{x}, R^{y}, m1, and m2 are as defined in formula (L-1), R^{v} is C₁-C₄ alkyl, halogen, or C₁-C₄ haloalkyl, and m4 is 0, 1, 2, 3, or 4.

In some embodiments, L is of formula (L-3a)

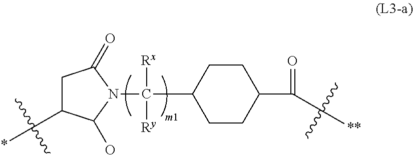

In a particular embodiment, L is of formula (L-3b)

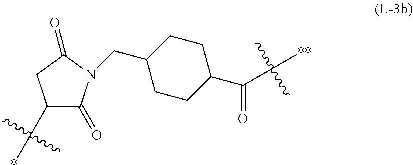

In some embodiments, Q¹ is —(CH₂CH₂O)ₘ₃— and L is of formula (L-4)

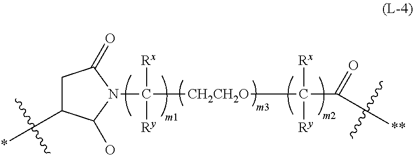

wherein R^{x}, R^{y}, m1, m2, and m3 are as defined in formula (L-1).

In some embodiments, L is of formula (L-4), wherein m3 is 1 or 2.

In some embodiments, L is of formula (L-4), wherein m3 is 1 or 2, and m1 is 0.

In some embodiments, L is of formula (L-4), wherein m3 is 1 or 2, m1 is 0, and m2 is 0, 1, or 2.

In some embodiments, L is of formula (L4-a) or (L4-b)

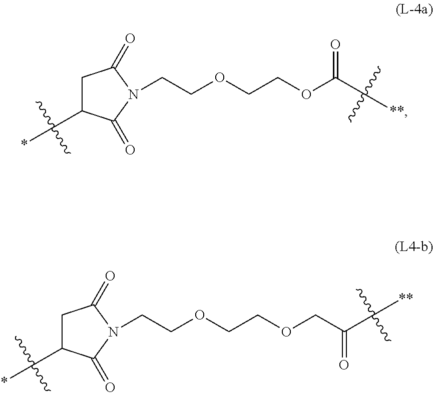

In some embodiments, Q² is -AAᵣ-, —Yₜ—, or -AAᵣ-Yₜ—, in which Y, if present, is attached to D, wherein AA, Y, r, and t are as defined in formula (L-1).

The -AAᵣ group may be a dipeptide, tripeptide, tetrapeptide, pentapeptide, hexapeptide, heptapeptide, octapeptide, nonapeptide, or decapeptide. Each “amino acid unit” independently may have a structure of formula (AA-1)

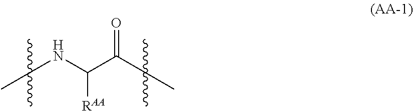

wherein R^{AA} is hydrogen, methyl, isopropyl, isobutyl, sec-butyl, benzyl, p-hydroxybenzyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-, phenyl, or cyclohexyl.

The amino acid units disclosed herein be enzymatically cleaved by one or more enzymes, including proteases, to liberate the drug unit (the D group). In some embodiments, the -AAᵣ-group is a valine-citrulline dipeptide have the following structure

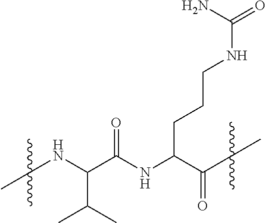 [SMILES: *NC(C(=O)NC(CCCNC(N)=O)C(*)=O)C(C)C]

The Y group at each occurrence is independently a self-immolative spacer unit. The self-immolative spacer unit may release the D group without the need for a separate hydrolysis step. In some embodiments, AA is present, Y is absent, and Q² is -AAᵣ-. In some embodiments, AA is absent, Y is present, and Q² is —Yₜ—. In some embodiments, both AA and Y are present, and Q² is-AAᵣ-Yₜ—. When Y is present, the Y moiety is attached to the D group.

In some embodiments, Y is a p-aminobenzyl alcohol (PAB) group. In some embodiments, Y is a PAB group that is linked to the AA moiety via the amino nitrogen atom of the PAB group, and connected directly to the D group, as shown below.

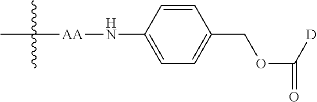

Other examples of self-immolative spacers include, but are not limited to, aromatic compounds that are electronically similar to the PAB group such as 2-aminoimidazol-5-methanol derivatives (see Hay et al., Bioorg. Med. Chem. Lett., 1999, 9, 2237) and ortho or para-aminobenzylacetals. Spacers can be used that undergo cyclization upon amide bond hydrolysis, such as substituted and unsubstituted 4-aminobutyric acid amides (Rodrigues et al., Chemistry Biology, 1995, 2, 223), appropriately substituted bicyclo[2.2.1] and bicyclo[2.2.2] ring systems (Storm, et al., J. Amer. Chem. Soc., 1972, 94, 5815) and 2-aminophenylpropionic acid amides (Amsberry, et al., J. Org. Chem., 1990, 55, 5867). In some embodiments, the self-immolative spacer unit contains a PAB unit and a cyclization module, which is connected to the D group, as shown below.

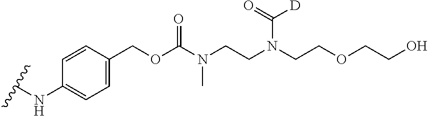 [SMILES: *Nc1ccc(COC(=O)N(C)CCN(CCOCCO)C([2H])=O)cc1]

Other suitable amino acid units and self-immolative spacer units include those disclosed in U.S. Pat. No. 7,829,531, which is incorporated herein by reference in its entirety.

2.3 Drug

The D group of formula (I) is a drug moiety. The drug moiety may include niclosamide and derivatives thereof. In some embodiments, the D group is of formula (D-1)

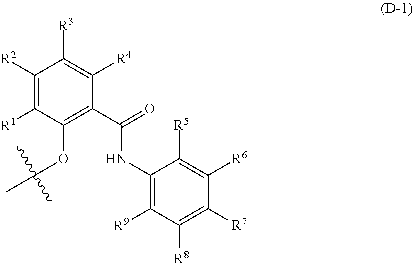

wherein

R¹, R², R³, R⁴, R⁵, R⁶, R⁸, and R⁹ at each occurrence are independently hydrogen, halogen, C₁-C₆ alkyl, C₁-C₆ haloalkyl, or C₁-C₆ alkoxy; and

R⁷ at each occurrence is halogen, C₁-C₆ haloalkyl, —NO₂, or —SO₂—C₁-C₄ alkyl.

In some embodiments, R⁷ is —NO₂. In some embodiment, R⁷ is —SO₂—C₁-C₄ alkyl, such as —SO₂CH₃. In some embodiments, R⁷ is C₁-C₆ haloalkyl, such as —CF₃.

In some embodiments, R³ is halogen. In some embodiments, R⁹ is halogen. In some embodiments, both R³ and R⁹ are halogen. In some embodiments, both R³ and R⁹ are Cl.

In some embodiments, the D group is of formula (D-2).

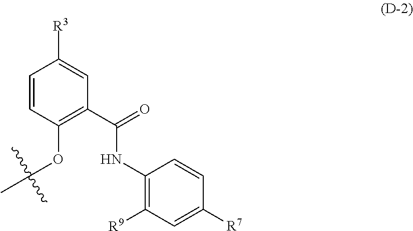

wherein R³, R⁷, and R⁹ are as defined in D-1.

In some embodiments, the D group is of formula (D-2), wherein R³ is halogen, R⁹ is halogen, and R⁷ is —NO₂, —SO₂CH₃, or —CF₃.

In some embodiments, the D group is a niclosamide moiety attached the L group. For example, the D group is of formula (D-3)

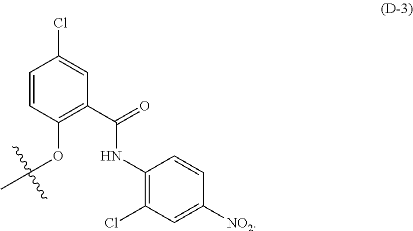 [SMILES: *Oc1ccc(Cl)cc1C(=O)Nc1ccc([N+](=O)[O-])cc1Cl]

Other suitable compounds that may be attached as the D group in the conjugate as disclosed herein include those described in U.S. Patent Application Publication No. 2013/0005802 to Chen et al. (“TREATMENT OF WNT/FRIZZLED-RELATED DISEASES,” filed Sep. 18, 2012), which is incorporated by reference herein in its entirety.

2.4 Structure of the Conjugate

In some embodiments, the -L-D group of the conjugate as disclosed herein has a structure resulting from any combination of a L group as described above (including, for example, formula (L-1), (L-2), (L-3), and (L-4)) and a D group as described above (including, for example, formula (D-1), (D-2), and (D-3)).

In some embodiments, the -L-D group has a structure, in which the L group is of formula (L-1) and the D group is of formula (D-1).

In some embodiments, the -L-D group has a structure, in which the L group is of formula (L-2) and the D group is of formula (D-1).

In some embodiments, the -L-D group has a structure, in which the L group is of formula (L-3) and the D group is of formula (D-1).

In some embodiments, the -L-D group has a structure, in which the L group is of formula (L-4) and the D group is of formula (D-1).

In some embodiments, the -L-D group has a structure, in which the L group is of formula (L-1) and the D group is of formula (D-3).

In some embodiments, the -L-D group has a structure, in which the L group is of formula (L-2) and the D group is of formula (D-3).

In some embodiments, the -L-D group has a structure, in which the L group is of formula (L-3) and the D group is of formula (D-3).

In some embodiments, the -L-D group has a structure, in which the L group is of formula (L-4) and the D group is of formula (D-3).

In some embodiments, the -L-D group has a structure of (LD-1), which includes a niclosamide moiety attached as the D group.

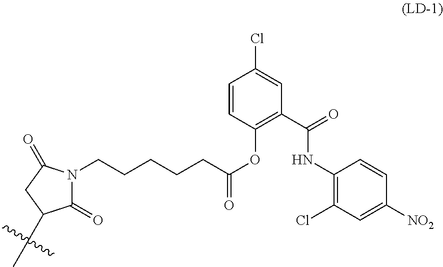 [SMILES: *C1CC(=O)N(CCCCCC(=O)Oc2ccc(Cl)cc2C(=O)Nc2ccc([N+](=O)[O-])cc2Cl)C1=O]

In some embodiments, the conjugate of formula (I) as disclosed herein contains one or more of the -L-D groups attached to the polypeptide. In some embodiments, the conjugate of formula (I) has 1, 2, 3, 4, 5, 6, 7, or 8-L-D groups attached to the polypeptide (p is 1, 2, 3, 4, 5, 6, 7, or 8, respectively). In some embodiments, the one or more -L-D groups of formula (I) are attached to the cysteine-enriched segment of the polypeptide via covalent bonds between the L moieties and the —SH groups in the cysteine residues of the cysteine-enriched segment.

In some embodiments, the conjugate of formula (I) has 4-L-D groups attached to the cysteine-enriched segment of the polypeptide via covalent bonds between the L moieties and the —SH groups in the cysteine residues of the cysteine-enriched segment. In some embodiments, disclosed herein is a population the conjugates of formula (I) having an average of approximately 4 drug molecules per chimeric polypeptide. In some embodiments, the attachment of approximately 4 drug molecules per chimeric polypeptide in a population of the conjugates as disclosed herein represents an amount of the attached (“loaded”) drug molecules at about 2 wt % of the resulting conjugates.

In some embodiments, the cysteine-enriched segment to which the one or more of -L-D groups of formula (I) are attached is located at the C-terminus of the polypeptide.

In some embodiments, the conjugate of formula (I) has a structure of formula (I-a), which includes a niclosamide moiety attached as the D group

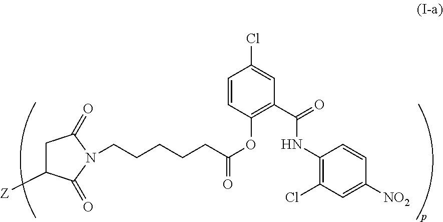

wherein Z and p are as defined in formula (I).

In some embodiments, the conjugate as disclosed herein has a structure of formula (I-a), wherein p is 1, 2, 3, 4, 5, 6, 7, or 8. In some embodiments, the conjugate as disclosed herein has a structure of formula (I-a), wherein p is 2, 3, 4, 5, 6. In some embodiments, the conjugate as disclosed herein has a structure of formula (I-a), wherein p is 4.

In some embodiments, the conjugate as disclosed herein has a structure of formula (I-a), wherein the -L-D groups are attached to the cysteine-enriched segment of the polypeptide via covalent bonds between the L moieties and the —SH groups in the cysteine residues of the cysteine-enriched segment.

In some embodiments, the polypeptide group (Z) is a genetically-encoded elastin-based chimeric polypeptide. The CP may consist of an elastin-like polypeptide fused to a short (Cys-Gly-Gly)₈ peptide segment that provides thiol reactive sites for chemical conjugation of chemotherapeutic drugs of interest. ELPs may be biopolymers comprising a Val-Pro-Gly-Xaa-Gly pentapeptide repeat. The “Xaa” may be any amino acid except Pro. The ELP may be derived from a structural motif found in mammalian elastin.

In some embodiments, the conjugate of formula (I) has a structure of formula (I-b)

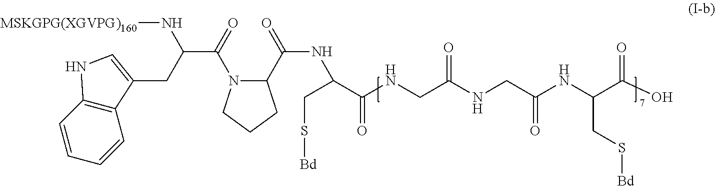

wherein X═V:G:A in a 1:7:8 ratio, and each Bd group is hydrogen or a -L-D group, in which the L and D groups are independently defined as above in the “Linker” and “Drug” sections.

In some embodiments, the conjugate has a structure of formula (I-b), wherein at least one Bd group is of formula (LD-1). In some embodiments, the conjugate has a structure of formula (I-b), wherein 2, 3, 4, 5, 6, 7, or 8 of the Bd groups are of formula (LD-1).

2.5 Linker-Drug Compound

In one aspect, provided herein are compounds that have as structural components the linker (L) and drug (D) moieties as disclosed here. In some embodiments, disclosed is a compound of formula (II), or a pharmaceutically acceptable salt thereof

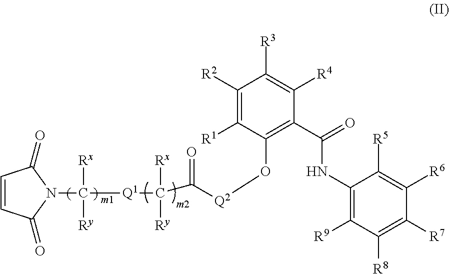

wherein,

Q¹ is bond, —O—, —NH—, aryl, cycloalkyl, heterocyclyl, heteroaryl, —(CH₂CH₂O)ₘ₃—, wherein the aryl, cycloalkyl, heterocyclyl, and heteroaryl are each optionally substituted by at least one C₁-C₄ alkyl, halogen, or C₁-C₄ haloalkyl;

Q² is bond, —O—CH(R^{w})—, -AAᵣ-, —Yₜ—, or -AAᵣ-Yₜ—, in which Y, if present, is attached to the

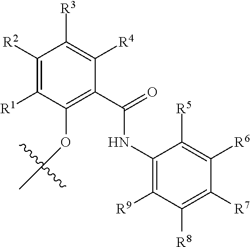

group;

R^{w} is H, C₁-C₆ alkyl, aryl, or cycloalkyl;

R^{x} and R^{y} at each occurrence are independently hydrogen or C₁-C₄ alkyl;

AA at each occurrence is independently an amino acid unit;

Y at each occurrence is independently a self-immolative spacer unit;

m1 is 0 to 10, provided that when Q1 is bond, m1 is 1-10;

m2 is 0 to 10;

m3 is 0 to 20;

r is 1-10;

t is 1 or 2;

R¹, R², R³, R⁴, R⁵, R⁶, R⁸, and R⁹ at each occurrence are independently hydrogen, halogen, C₁-C₆ alkyl, C₁-C₆ haloalkyl, or C₁-C₆ alkoxy; and

R⁷ at each occurrence is halogen, C₁-C₆ haloalkyl, —NO₂, or —SO₂—C₁-C₄ alkyl.

In some embodiments, the compound is of formula (II), wherein Q² is bond.

In some embodiments, the compound is of formula (II), wherein Q² is bond and Q¹ is bond.

In some embodiments, the compound is of formula (II-a), or a pharmaceutically acceptable salt thereof

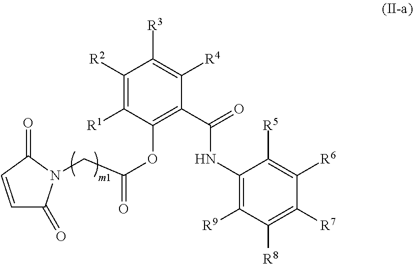

wherein m1, R¹, R², R³, R⁴, R⁵, R⁶, R⁷, R⁸, and R⁹ are as defined in formula (II).

In some embodiments, the compound is of formula (II), wherein Q² is bond and Q¹ is an optionally substituted cycloalkyl, aryl, or —(CH₂CH₂O)ₘ₃—. In some embodiments, the compound is of formula (II-b), or a pharmaceutically acceptable salt thereof

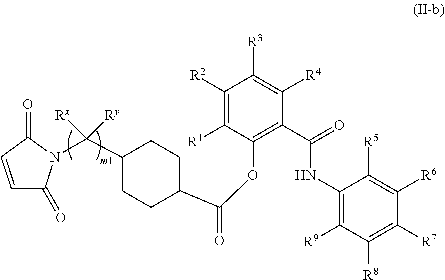

In some embodiments, the compound is of formula (II), or a pharmaceutically acceptable salt thereof, wherein Q² is -AAᵣ, —Yₜ—, or -AAᵣ-Yₜ—, in which Y, if present, is attached to the

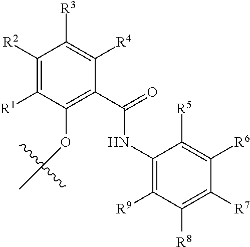

group.

The AA groups are each an amino acid unit as described above. The Y groups are each a self-immolative spacer unit as described above.

In some embodiments, R⁷ is —NO₂. In some embodiment, R⁷ is —SO₂—C₁-C₄ alkyl, such as —SO₂CH₃. In some embodiments, R⁷ is C₁-C₆ haloalkyl, such as —CF₃.

In some embodiments, R³ is halogen. In some embodiments, R⁹ is halogen. In some embodiments, both R³ and R⁹ are halogen. In some embodiments, both R³ and R⁹ are Cl.

In some embodiments, R¹, R², R⁴, R⁵, R⁶, and R⁸ are hydrogen.

In some embodiments, R¹, R², R⁴, R⁵, R⁶, and R⁸ are hydrogen, R⁷ is —NO₂, —SO₂CH₃, or —CF₃.

In some embodiments, R¹, R², R⁴, R⁵, R⁶, and R⁸ are hydrogen, R³ is halogen, and R⁹ is halogen.

In some embodiments, R¹, R², R⁴, R⁵, R⁶, and R⁸ are hydrogen, R³ is halogen, R⁹ is halogen, and R⁷ is —NO₂, —SO₂CH₃, or —CF₃. In some embodiments, R¹, R², R⁴, R⁵, R⁶, and R⁸ are hydrogen, R³ is halogen, R⁹ is halogen, and R⁷ is —NO₂.

In some embodiments, the compound of formula (II) includes niclosamide as a drug moiety. For example, the compound of formula (II) may have a structure of

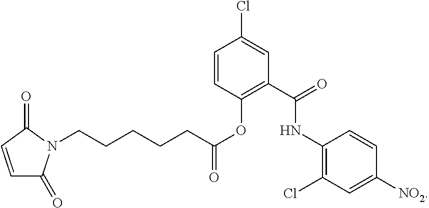 [SMILES: O=C(CCCCCN1C(=O)C=CC1=O)Oc1ccc(Cl)cc1C(=O)Nc1ccc([N+](=O)[O-])cc1Cl]

2.6 Synthesis of the Conjugate.

The linker-drug compounds of formula (II) are useful for preparing a conjugates, such as certain conjugates disclosed herein, in a conjugation reaction. The compounds as disclosed herein have a maleimide group that may react with the sulfhydryl (—SH) group on a polypetide to form a covalent bond, thereby attaching the compound to the polypeptide through the covalent bond.

In one aspect, provided herein is a method of preparing a conjugate, the method comprising the steps of preparing a polypeptide having a cysteine-enriched segment; and reacting the polypeptide with a compound of formula (II) or a pharmaceutically acceptable salt thereof to form the conjugate, wherein the compound is covalently attached to the cysteine-enriched segment of the polypeptide.

In some embodiments, the polypeptide is an elastin-like polypeptide. In some embodiments, the cysteine-enriched segment is at the C-terminus of the polypeptide. In some embodiments, the cysteine-enriched segment comprises (Gly-Gly-Cys)ₙ, wherein n is 2 to 10.

The CP may be expressed from a plasmid-borne synthetic gene in E. coli and purified by inverse transition cycling (ITC), using temperature-dependent self-assembly phase shift.

In some embodiments, the compound used in the preparation method disclosed herein is a compound of formula (II-a) or a pharmaceutically acceptable salt thereof. In some embodiments, the compound used in the preparation method disclosed herein is a compound of formula (II-b) or a pharmaceutically acceptable salt thereof.

In some embodiments, the compound used in the preparation method disclosed herein is a compound of formula

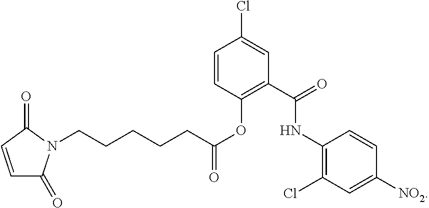 [SMILES: O=C(CCCCCN1C(=O)C=CC1=O)Oc1ccc(Cl)cc1C(=O)Nc1ccc([N+](=O)[O-])cc1Cl]

In some embodiments, the conjugation reaction may achieve an amount of the “loaded” drug molecules at about 2 wt % of the resulting conjugates. For some conjugates (such as ELP-based conjugates), the amount of about 2% drug loading may correspond to approximately 4 drug molecules per chimeric polypeptide. The term “drug loading” as used herein refers to the weight percentage of the attached drug molecules in the conjugates as disclosed herein. The drug loading values may be determined from the mass difference between the conjugate and the unconjugated polypeptide, as measured by known technologies, such as matrix-assisted laser desorption/ionization, time-of-flight mass spectrometer (MALDI-TOF MS).

In some embodiments, the polypeptide may be reduced by a reducing agent prior to the conjugation reaction. Typically, the polypeptide is mixed with the compound of formula (II) in a reaction buffer, at pH about 7.0. The mixture may be stirred at about room temperature (such as 20-25° C.) for a period of time to allow the formation of covalently bonds between the molecules of the compound and the —SH groups in the cysteine-enriched segment of the polypeptide.

Following the conjugation reaction, the conjugate prepared by the method disclosed herein may be purified by known techniques. In some embodiments, the conjugate may remain in the supernatant, and may be isolated by centrifugation and subsequent removal of solvent (such as by freeze drying).

### 3. Nanoparticles of the Conjugate

In some embodiments, the conjugate as disclosed herein may form nanoparticles through self-assembly. Without being bound by any theory, it is hypothesized that the hydrophobic moieties of the conjugates as disclosed herein may be physically encapsulated into a micelle formed by the polypeptide chains of the conjugates.

In one aspect, disclosed here is a nanoparticle comprising the conjugate of formula (I) or a pharmaceutically acceptable salt thereof, wherein the -D groups of the conjugate form a core of the nanoparticle.

In some embodiments, a plurality of the conjugates as disclosed herein may undergo self-assembly in an aqueous medium to form a cylindrical nanoparticle, which includes a core formed by an aggregation of the hydrophobic drug moieties of the plurality of conjugates, and a hydrophilic portion formed by the polypeptides of the conjugates surrounding the core.

In some embodiments, the nanoparticles as disclosed herein may have a critical micelle concentration (CMC) of from about 0.5 μM to about 5.0 μM, including from about 1 μM to about 4.5 μM, from about 1.5 μM to about 4.0 μM, from about 2.0 μM to about 3.5 μM, from about 2.8 μM to about 3.2 μM. In some embodiments, the nanoparticles as disclosed herein may have a CMC of about 2.0 μM, about 2.5 μM, about 3.0 μM, or about 3.5 μM.

In some embodiments, the aggregation number of the nanoparticles disclosed herein may range from about 50 to about 200, including from about 50 to 150, from about 50 to about 100, and from about 80 to about 100. In some embodiments, the aggregation number of the nanoparticles disclosed herein may be about 80, about 90, or about 100.

In some embodiments, the radius of gyration (Rg) of the nanoparticles disclosed herein may range from about 40 nm to about 150 nm, including from about 50 nm to about 140 nm, from about 60 nm to about 120 nm, from about 65 nm to about 100 nm, from about 70 nm to about 90 nm, and from about 75 nm to about 85 nm. In some embodiments, the Rg of the nanoparticles disclosed herein may be about 65 nm, about 70 nm, about 75 nm, about 80 nm, about 85 nm, about 90 nm, about 95 nm, or about 100 nm.

In some embodiments, the experimentally determined form factor (p), calculated as Rg/Rh (hydrodynamic radius), of the nanoparticles disclosed herein may be about 1.2, about 1.3, about 1.4, about 1.5, about 1.6, about 1.7, about 1.8, about 1.9, or about 2.0.

In some embodiments, the average length of the disclosed nanoparticle as determined by cryo-TEM (LTEM) may range from about 50 nm to about 100 nm, including from about 60 nm to about 90 nm, and from about 70 nm to about 80 nm. In some embodiments the average diameter as determined by DTEM may range from about 5 nm to about 20 nm, including from about 7 nm to about 18 nm, from about 9 nm to about 16 nm, from about 10 nm to about 15 nm, from about 11 nm to 14 nm, from about 10 nm to about 13 nm.

In some embodiments, the nanoparticles as disclosed herein may have a micellar morphologies as verified by atomic force microscopy (AFM) under ambient condition. In some embodiment, the nanoparticles as disclosed herein may show a rod or worm-like morphology according to AFM images.

In some embodiments, the nanoparticles as disclosed herein may have an inverse transition temperature (Tt) that is independent of the conjugate concentration in the range of 5-50 μM. Without be limited by any theory, it was hypothesized that the local polypeptide concentration in the nanoparticles makes the Tt nearly independent of the conjugate's overall, solution concentration.

### 4. Compositions

Also provided a pharmaceutical composition comprising the conjugate of formula (I) or a pharmaceutically acceptable salt thereof and a pharmaceutically acceptable carrier.

In some embodiments, the conjugates as disclosed herein may be formulated into a composition in accordance with standard techniques well known to those skilled in the pharmaceutical art. The composition may be prepared for administration to a subject. Such compositions comprising a conjugate can be administered in dosages and by techniques well known to those skilled in the medical arts taking into consideration such factors as the age, sex, weight, and condition of the particular subject, and the route of administration.

The pharmaceutical compositions may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

In some embodiments, the pharmaceutical composition includes nanoparticles of the conjugates. The nanoparticles may be formed, for example, by aggregation of a plurality of the conjugates. In some embodiments, the pharmaceutical composition includes nanoparticles of the conjugates as disclosed herein, which includes a core formed by an aggregation of the hydrophobic drug moieties of the plurality of conjugates, and a hydrophilic portion formed by the polypeptides of the conjugates surrounding the core. In some embodiments, the pharmaceutical composition include nanoparticles of the conjugates as disclosed herein, and the nanoparticles demonstrate one of more physical properties, including CMC, Rg, form factor (Rg/Rh), length, diameter, micellar morphologies, and inverse transition temperature, as disclosed herein.

### 5. Method of Treating a Disease

Also provided is a method for treating a disease, comprising administering to a subject in need thereof an effective amount of a pharmaceutical composition comprising the conjugate of formula (I) as disclosed herein or a pharmaceutically acceptable salt thereof and a pharmaceutically acceptable carrier.

In some embodiments, the disease is cancer, parasite infection, bacterial infection, viral infection, metabolic diseases, Type II diabetes, NASH, NAFLD, artery constriction, endometriosis, neuropathic pain, rheumatoid arthritis, sclerodermatous graft-versus-host disease, and/or systemic sclerosis.

In some embodiments, the disease is a Wnt/Frizzled-related disease. A “Wnt/Frizzled-related disease,” as used herein, is a disease in which the Wnt/Frizzled signaling pathway is dysregulated. Certain exemplary Wnt/Frizzled-related diseases include, but are not limited to, cardiovascular disease, neoplasm, obesity, osteoporosis, neuron degeneration, cancer, and disorders in wound healing and tissue repair. The Wnt/Frizzled signaling pathway may be considered dysregulated when, for example, diseased tissue and/or cells comprise at least one of: increased levels of β-catenin; increased LEF/TCF-mediated transcription; increased levels of one or more Wnt proteins, including, but not limited to, Wnt3A; increased levels of Frizzled; and/or increased levels of Dishevelled; as compared to normal tissue and/or cells. As used herein, the term “tissue” includes all biological tissues, including, but not limited to, organ tissue, tumor tissue, skin, blood, etc.

In some embodiments, a Wnt/Frizzled-related disease is a cardiovascular disease, such as myocardial infarction and cardiac hypertrophy. Cardiovascular disease may further include coronary heart disease (including heart attack and angina pectoris or chest pain); stroke; hypertension, high blood pressure; heart failure; rheumatic fever/rheumatic heart disease; congenital cardiovascular defects; arrhythmias (disorders of heart rhythm); diseases of the arteries, arterioles, and capillaries (including atherosclerosis and Kawasaki disease); bacterial endocarditis; cardiomyopathy; valvular heart disease; diseases of pulmonary circulation; diseases of veins and lymphatics; and other diseases of the circulatory system. In certain embodiments, inhibition of Wnt signaling in such cardiovascular diseases results in a beneficial effect on infarct healing, increased angiogenesis, and/or an attenuated hypertrophic response in the heart.

In some embodiments, a Wnt/Frizzled-related disease is a neoplasm. In certain embodiments, neoplasm is cancer or a cancer cell. Certain exemplary Wnt/Frizzled-related cancers include, but are not limited to, colon cancer, melanomas, hepatocellular carcinomas, leukemia, ovarian cancer, prostate cancer, lung cancer, brain tumor, and breast cancer.

The embodiments, provided is a method of treating cancer selected from colon cancer, melanomas, hepatocellular carcinomas, leukemia, ovarian cancer, prostate cancer, lung cancer, brain tumor, and breast cancer in a subject, which includes administering to the subject an effective amount of a pharmaceutical composition comprising the conjugate of formula (I) or a pharmaceutically acceptable salt thereof and a pharmaceutically acceptable carrier.

In some embodiments, the conjugate may be administered prophylactically or therapeutically. In prophylactic administration, the conjugate can be administered in an amount sufficient to induce a response. In therapeutic applications, the conjugates are administered to a subject in need thereof in an amount sufficient to elicit a therapeutic effect. Effective amounts of the conjugate may depend on, for example, the particular composition of the conjugate regimen administered, the manner of administration, the stage and severity of the disease, the general state of health of the patient, and the judgment of the prescribing physician.

Niclosamide has been shown to exert anti-proliferative effects in human colon cancer cell lines by inhibiting Wnt/β-catenin pathway activation, down-regulating Dvl2 and reducing downstream β-catenin signaling. Niclosamide is poorly absorbed and metabolizes rapidly in vivo, and it remains a challenge to deliver niclosamide to a subject while maintaining minimal side effects, acceptable pharmacological properties, and effective anti-tumor activities. Advantageously, the conjugation of multiple copies of a drug molecule (such as niclosamide) to a chimeric polypeptide as disclosed herein triggers self-assembly of the conjugates into cylindrical nanoparticles, which may be highly water soluble. These nanoparticles may show comparable inhibition of β-catenin expression and growth of HCT-116 cells as free drug, and when administered intravenously, liberate the drug compound (such as niclosamide) with better systemic distribution and better anti-tumor in vivo efficacy compared to direct dosing with free drug.

In some embodiments, the conjugate as disclosed herein may allow for precise control over the location of the drug release in endosomes. Advantageously, the conjugates as disclosed herein may demonstrate more effective in vivo tumor regression as compared to physically encapsulated drugs (such as those encapsulated in polymeric micelles).

In addition to therapeutic efficacy, the conjugates and nanoparticles as disclosed herein may have several other useful characteristics compared to synthetic polymeric micelles and polymer conjugates that require complicated multistep procedures to synthesize. For example, the recombinant polypeptides as disclosed herein may be synthesized in E. coli (or other expression systems) with high yield and purified readily using phase-shift coacervation, allowing complete control of their molecular weight and polydispersity. They are biodegradable and self-assemble in aqueous buffer into nearly monodisperse nanoparticles upon conjugation with niclosamide or other small hydrophobic drugs. In some embodiments, attachment of hydrophobic drugs solely at the chain end (such as C-terminus) may ensure that the drug is sequestered within the nanoparticle core, unlike other nanoparticle drug carriers, such as dendrimers, metal nanoparticles or carbon nanotubes that expose the hydrophobic drugs at the nanoparticle-water interface.

Accordingly, the compositions disclosed herein may be used to treat is cancer, parasite infection, bacterial infection, viral infection, metabolic diseases, Type II diabetes, NASH, NAFLD, artery constriction, endometriosis, neuropathic pain, rheumatoid arthritis, sclerodermatous graft-versus-host disease, systemic sclerosis, or combinations thereof.

### 6. Examples

### Example 1. Materials and Methods

Static and Dynamic Light Scattering.

Dynamic light scattering (DLS) was used to measure the particle size at 25° C. and at 10 μM concentration (n=3) in PBS after filtration through an Anotop syringe filter with 0.22 μm size pores (Whatman; Florham Park, N.J.) using a DynaPro Plate Reader (Wyatt Technology; Santa Barbara, Calif.). To obtain size histograms, regularization fits were used to determine the hydrodynamic radius (Rh) as weighted by the percent by mass. Static and dynamic light scattering (SLS/DLS) measurements were performed on an ALV/CGS-3 goniometer system (Langen, Germany). Samples for the ALV/CGS-3 goniometer system were prepared in PBS and filtered through 0.22 μm Millex-GV filters into a 10 mm disposable borosilicate glass tube (Fisher). Simultaneous SLS and DLS measurements were obtained at 22° C. for angles between 30°-150° at 5° increments, with measurements at each angle consisting of 3 runs for 15 seconds. The differential refractive index (dn/dc) was determined by measuring the refractive index at five different concentrations using an Abbemat 500 refractometer (Anton Paar, Graz, Austria). DLS data were analyzed by fitting the autocorrelation function to a biexponential decay using the HDRC software package (Germany). Rh was plotted against angle and extrapolated to zero. SLS data were analyzed by partial Zimm plots using ALV/Dynamic and Static FIT and PLOT software in order to determine the radius of gyration and molecular weight.

Cryogenic Transmission Electron Microscopy.

Cryogenic transmission electron microscopy (cryo-TEM) was performed at Duke University's Shared Materials Instrumentation Facility (Durham, N.C.). Lacey holey carbon grids (Ted Pella, Redding, Calif.) were glow discharged in a PELCO EasiGlow Cleaning System (Ted Pella, Redding, Calif.). A 3 μl drop of a sample was deposited onto the grid, blotted for 3 s with an offset of −3 mm, and vitrified in liquid ethane using the Vitrobot Mark III (FEI, Eindhoven, Netherlands). Prior to vitrification, the sample chamber was maintained at 22° C. and 100% relative humidity to prevent sample evaporation. Grids were transferred to a Gatan 626 cryoholder (Gatan, Pleasanton, Calif.) and imaged on a FEI Tecnai G2 Twin TEM (FEI, Eindhoven, Netherlands).

Atomic-Force Microscopy (AFM).

Samples for AFM imaging were prepared by placing a drop of sample solution (˜0.2 mg/ml) onto a freshly cleaved mica surfaces and incubating for 15 minutes. Then, the sample was gently rinsed with Milli-Q H2O and dried under a N2 stream. All AFM images were acquired with Tapping Mode under ambient conditions using a MultiMode AFM (Bruker). TappingMode silicon cantilever was used for all the AFM images (kF=40 N/m, fres=300 kHz).

Western Blot.

Western blots were performed following a procedure similar to that reported previously (Osada et al., Cancer Res., 2011, 71, 4172-4182). Briefly, HCT-116 cells were grown to about 80% confluency on poly-D-lysine coated six-well plates for 48 and then incubated with 2.5 μM NIC in DMSO, molar equivalent CP—NIC or DMSO control for 18 hours in growth medium. After treatment, the cytosolic fraction was isolated as previously described (Chen et al., Biochemistry, 2009, 48, 10267-10274). Immunoblot was used to detect the 3-catenin, c-myc and cyclin D1 protein levels in cytosol, with β-actin immunoblots used for loading control.

Pharmacokinetic Analysis of CP—NIC.

CP—NIC was dissolved in PBS at a concentration of 40 mg/ml and injected i.v. in the tail vein of CD1 mice at a dose of 128 mg/kg of body weight. Blood samples were obtained at 0.5 h prior to the dosing and at 0.08, 0.17, 0.33, 0.67, 1.5, 4, 8, 12, and 24 h after drug administration. Quantification of free NIC in mouse plasma was done by LC/MS-MS using methods similar to those previously published.

In Vitro Cell Proliferation.

HCT116 human colon carcinoma cells were purchased from American Type Culture Collection (Manassas, Va., USA) and maintained in McCoy's 5A medium supplemented with 10% fetal bovine serum (FBS, Atlanta Biologicals, Lawrenceville, Ga., USA), 200 U/ml penicillin, and 50 ng/ml streptomycin (Invitrogen, Grand island, NY, USA). Cells were grown at 37° C. in 5% CO₂. The cells were plated at 5,000 cells per well into 96 well plates and treated with compounds (n=3) for 48 hours, at which point the cell proliferation was measured using the colorimetric MTS assay (Promega, Madison, Wis., USA). Values were normalized as a percentage of DMSO treated cells. The dose response data were fit with sigmoidal dose-response using Graphpad Prism.

Dose Escalation and Tumor Inhibition.

Prior to in vivo implantation, HCT116 cells were washed twice in Minimum Essential Media (MEM) (51200-038; Invitrogen; Carlsbad, Calif.). HCT116 cells were implanted in the right flank of male nude mice by subcutaneous injection of 1-2×106 cells in 50 μL. All animals were treated in accordance with National Institute of Health Guide for the Care and Use of Laboratory Animals under protocols approved by the Duke University Institutional Animal Care and Use Committee.

Male nude mice (6-8 weeks old) bearing subcutaneous HCT116 tumors were treated when the mice had a tumor volume of 75-100 mm³. Controls or drugs were administered by tail vein infusion (50 μL/min) of 500 μL. Dose escalation was performed with CP—NIC at 5, 10, 15, 20, and 25 mg/kg BW (BW: body weight). Mice were treated 3 days/week for 2 weeks with either 5 mg/kg BW unconjugated NIC, or 20 mg/kg BW CP—NIC, the maximum tolerated doses, respectively. Tumor dimensions and BW were measured 3-4 times a week, and the tumor volume was calculated according to Volume [mm³]=length×width×depth×½.

Mice were monitored for BW loss, and euthanized upon exceeding 15% loss in BW or if their tumors grew to a volume greater than 1000 mm3. The maximum tolerated dose (MTD) was determined in mice with tumors. Cumulative survival curves were compared using Kaplan-Meier analysis, and the Sidak test, Tukey Test and Wilcoxon test were calculated using GraphPad Prism 6 software.

Synthesis of Chimeric Polypeptides.

The CP used for conjugation to NIC consists of the sequence SKGPG-(XGVPG)₁₆₀-WPC(GGC)₇ (single amino acid codes), where the guest residue X═V:G:A in a 1:7:8 ratio.

The CP was expressed from a plasmid-borne synthetic gene in E. coli and purified by inverse transition cycling (ITC), using temperature-dependent self-assembly phase shift. Three rounds of ITC yielded 100 mg of purified monodisperse CP from 1 L of culture. Specifically, the CP was expressed from a pET-24b expression plasmid transformed into Escherichia coli strain BL21(DE3), using a previously published hyperexpression protocol that relies on the leakiness of the T7 promoter (Chilkoti et al., Nature biotechnology, 1999; 17:1112-5). Six 50 mL cultures grown for 16 h were used to inoculate six 1 L flasks of TB dry supplemented with 45 μg/mL kanamycin. Each 1 L flask was incubated for 24 h at 37° C. at 210 rpm, and the cell pellet collected by centrifugation at 3,000 rpm for 10 min at 4° C. CP was purified using inverse transition cycling (ITC), a non-chromatographic purification method that exploits the temperature-dependent phase transition of CPs (MacKay et al., Nature materials, 2009; 8:993-9). Briefly, the cell pellet was resuspended in PBS and lysed via sonication on ice for 3 min (10 s on, 40 s off) (Misonix S-4000; Farmingdale, N.Y.). Polyethyleneimine (PEI) 0.7% w/v was added to the lysate to precipitate nucleic acid contaminants. The supernatant was then subjected to repeated rounds of ITC as follows: the solution was heated to 37° C. in the presence of 3 M NaCl to induce coacervation, then centrifuged for 10 min at 14,000 g and 20° C., and the pellet resuspended in 20 mM TCEP in water, pH 7. This suspension was cooled to 4° C. to induce dissolution of aggregates, and then centrifuged for 10 min at 14,000 and 4° C. to remove any insoluble contaminants. Typically, three rounds of ITC generated a pure product (>95% by SDS-PAGE). Proteins were visualized by Simply Blue Safe Stain (Invitrogen, LC6060) (Steinberg, Methods in enzymology, 2009; 463:541-63). In brief, proteins were separated on 10% SDS-PAGE mini-gel. After electrophoretic separation, the mini-gel was rinsed with 100 m1 ultrapure water 3 times for 5 minutes, followed by staining with Simply Blue Safe Stain for at least 1 hour at room temperature with gentle shaking.

Determination of NIC Conjugation Ratio.

The conjugation ratio of NIC to CP was determined by matrix-assisted laser desorption/ionization time-of-flight mass spectrometry (MALDI-TOF-MS) of the CP—NIC conjugate and free CP using a Voyager DE-Pro MALDI-MS (Applied Biosystems) instrument equipped with a nitrogen laser (337 nm). The MALDI-TOF-MS samples were prepared in an aqueous 50% acetonitrile solution containing 0.1% trifluoroacetic acid (TFA), using a sinapinic acid matrix. The conjugation ratio was determined by examining the increase in mass of the CP—NIC conjugate relative to unmodified CP.

Temperature Programmed Turbidimetry.

The transition temperature (Tₜ) of each sample was calculated by recording the optical density at 650 nm as a function of temperature (1° C./min ramp) on a temperature controlled UV-Vis spectrophotometer (Cary 300 Bio; Varian Instruments, Palo Alto, Calif.). The Tₜ was defined as the inflection point of the turbidity profile. All samples were analyzed in 90% mouse serum with CP concentrations in the range of 5-50 μM.

Determination of CMC.

CP—NIC was characterized by fluorescence spectroscopy using pyrene as a probe of local hydrophobicity, which enables measurement of the critical micelle concentration (CMC) of CP—NIC micelles. The ratio of the first fluorescence emission peak (I₃₇₀₋₃₇₃) and the third peak (I₃₈₁₋₃₈₄) were plotted over a range of CP—NIC concentrations. The sigmoid of best fit was used to calculate the CMC, defined as the inflection point of the curve.

### Example 2. Synthesis of CP—NIC Conjugate

The synthesis of a representative CP—NIC conjugate was carried out by a process according to Scheme 1. A terminal maleimide was added to NIC via a substituted hexanoic acid to enable conjugation of NIC to the polypeptide. Treatment of NIC with 6-Maleimidohexanoic acid and N,N′-dicyclohexylcarbodiimide (DCC) produced the 6-Maleimidohexanoic ester derivative of NIC (I), which was covalently attached to the Cys residues of the CP.

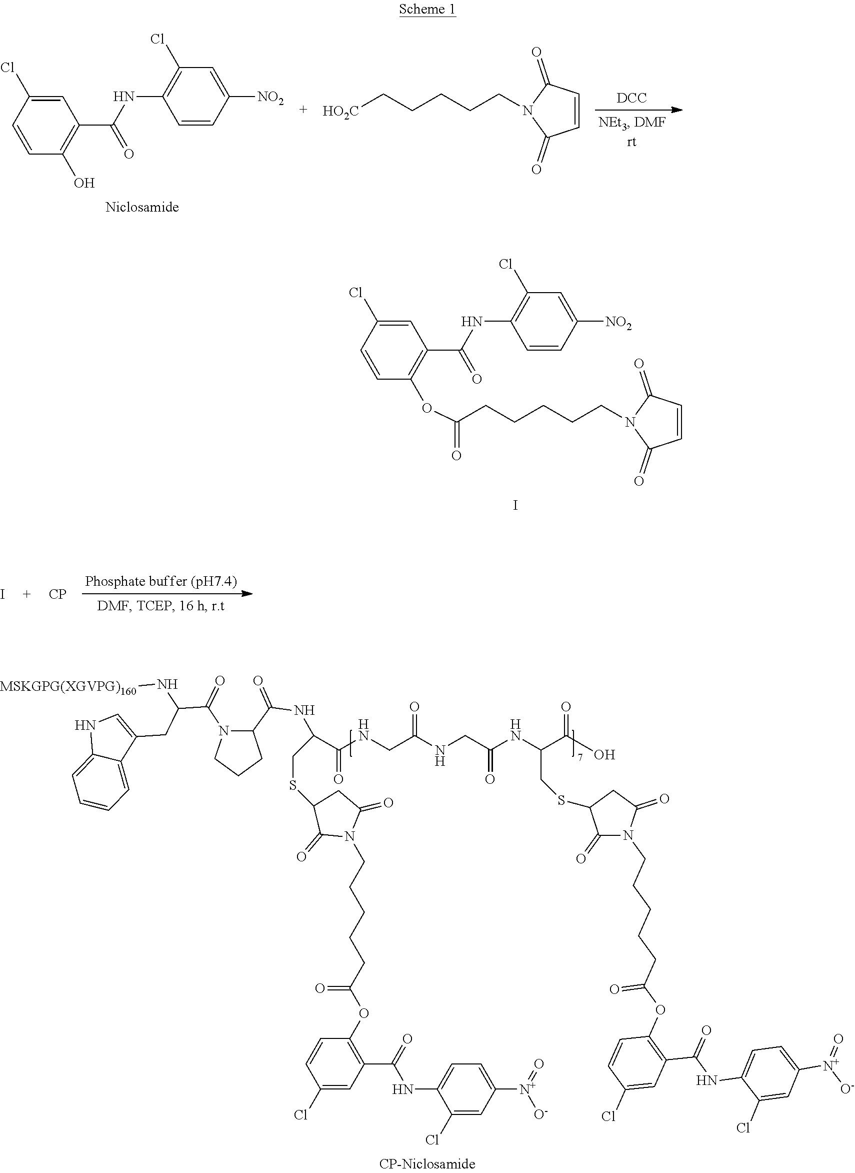

Specifically, NIC (1.032 g, 3.16 mmol) and dry DMF (5 mL) were added to a dry vial. Next, Et₃N (0.4 mL, 2.84 mmol) was added to the suspension, and the mixture was sonicated to produce a red-colored homogeneous solution. DCC (1.95 g, 9.47 mmol), dry DMF (5 mL) and 6-Maleimidohexanoic acid (1.99 g, 9.47 mmol) were added to a dry round-bottomed flask equipped with a magnetic stir bar under an Argon atmosphere. The red DMF suspension of NIC was added dropwise over 2 min to this solution at room temperature, and the vial was rinsed with twice with 1 mL dry DMF that was added to the flask. After 5% hours, an additional 0.2 mL of Et₃N was added, the reaction mixture was stirred for an additional 15 min, and then filtered to remove a white precipitate that formed during the course of the reaction. The filtrate was poured into 200 mL of 0.1 M NaH₂PO₄ solution at pH 4-5, and the mixture was extracted twice by 75 mL ethyl acetate. The ethyl acetate solutions were combined and washed three times with PBS (pH 4-5), three times with water, twice with 3% sodium bicarbonate (freshly prepared), once with saturated sodium chloride solution, then dried over sodium sulfate and filtered. To the filtrate was added 5 mL of silica gel, and ca. 25 mL heptane, and the mixture was concentrated to dryness on a rotary evaporator. The solids were loaded onto a 120 mL silica gel column packed in 1% ethyl acetate/chloroform, and eluted with a gradient of 1-4% ethyl acetate/chloroform. The fractions containing the desired material (R_{f}=0.2 in 4% EtOAc/CHCl₃) were combined and concentrated to give 1.34 g (82%) of the 6-Maleimidohexanoic ester of NIC (4-chloro-2-((2-chloro-4-nitrophenyl)carbamoyl)phenyl 6-(2,5-dioxo-2,5-dihydro-1H-pyrrol-1-yl)hexanoate) as a pale yellow solid. ¹H NMR (400 MHz, DMSO-d₆) δ=10.44 (br. s, 1H), 8.36 (d, J=2.54 Hz, 1H), 8.23 (dd, J=2.60, 9.20 Hz, 1H), 8.05 (d, J=9.08 Hz, 1H), 7.79 (d, J=2.54 Hz, 1H), 7.66 (dd, J=2.54, 8.7 Hz, 1H), 7.30 (d, J=8.7 Hz, 1H), 6.96 (br. s, 2H), 3.28 (t, J=7.2 Hz, partial overlap with H2O peak), 2.50 (t, J=7.3 Hz partial overlap with DMSO peak), 1.47-1.63 (m, 2H), 1.30-1.45 (m, 2H), 1.12-1.26 (m, 2H). MS (ESI) m/z=518 (M−1). FTIR (thin film, cm⁻¹) u=3369 (br, med), 1770 (med), 1702 (st).

Prior to conjugation with the 6-Maleimidohexanoic ester of NIC (compound I), purified CP was suspended in reaction buffer (0.1 M sodium phosphate, 1 mM Ethylenediaminetetraacetic acid (EDTA), pH 7.0) and reduced with 1 mL of Tris(2-carboxyethyl)phosphine hydrochloride (TCEP) at neutral pH (100 mM, pH 7.0) at ˜5× excess to thiol. Excess TCEP was removed from the solution by initiating the phase transition with sodium chloride (2.5 M) and centrifugation at 4,000 rpm at 25° C. for 10 minutes. The CP pellet obtained by centrifugation was re-suspended in ˜2 mL of reaction buffer. 6-Maleimidohexanoic ester of NIC (compound I). Purified Nic-ε-maleimidocaproic acid (NIC-EMCA was suspended in ˜2 mL of DMF and slowly transferred to the stirring CP solution. 1 mL of pH neutral TCEP (100 mM) was added and the reactants were stirred for 16 hrs at 20° C. in the dark. After reaction, the unreacted Niclosamide 6-Maleimidohexanoic ester precipitate was separated by centrifugation at 13,000 rpm at 10° C. for 10 minutes. The supernatant was further purified by diluting it in 20% acetonitrile in PBS and centrifuging the solution in an Amicon Ultra-15 Centrifugal Filter Units (MWCO: 10 KDa, Millipore) at 2,500 rpm at 10° C. The CP—NIC solution was washed twice with NH₄HCO₃ solution (pH 7.4) and then freeze-dried.

### Example 3. Characterization of CP—NIC Conjugate

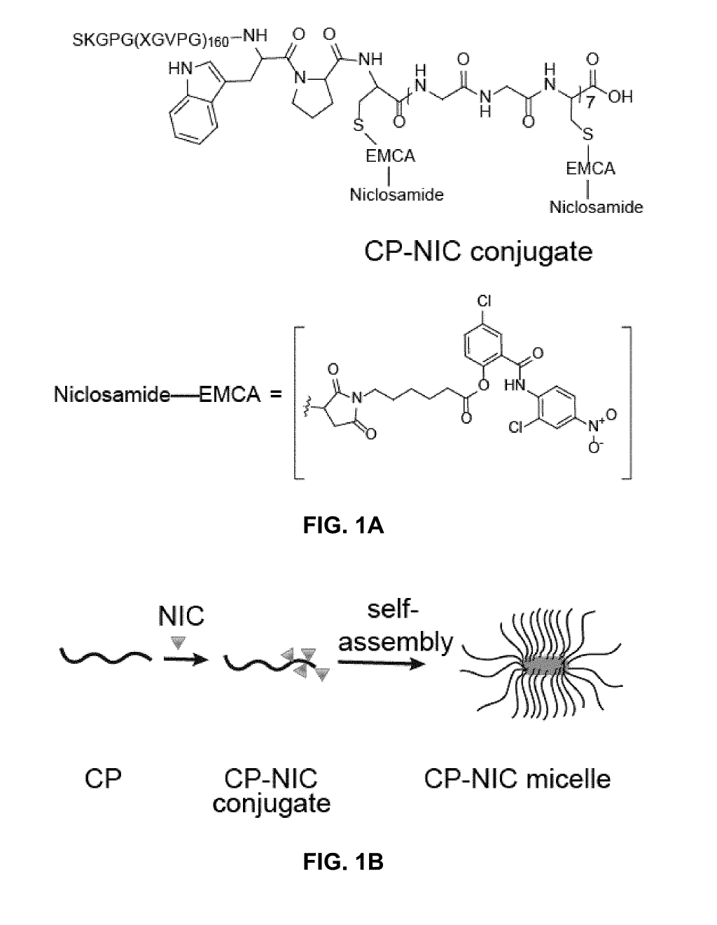

A representative chimeric polypeptide-niclosamide (CP—NIC) conjugate was prepared to explore the advantage of nano-formulation technology to deliver NIC as a targeted therapeutic agent with improved pharmacodynamic properties (FIGS. 1A-1B). Specifically, a representative CP prepared here included an elastin-like polypeptide (ELP), a disordered and highly water soluble recombinant peptide polymer, and a Cys-(Gly-Gly-Cys)₇ peptide segment at the C-terminus (FIG. 1A). The CP was conjugated to NIC through the covalent bonding between the C-terminus Cys residues of the CP and the maleimide group of a 6-maleimidohexanoic ester derivative of NIC (FIG. 1A). It was observed that the attachment of NIC as a hydrophobic moiety to the hydrophilic polypeptide chain triggers self-assembly of the CP—NIC conjugate into cylindrical nanoparticles with a drug-rich core (formed by the aggregation of the hydrophobic NIC moieties) and surrounding hydrophilic polypeptide chains from CP (FIG. 1B).

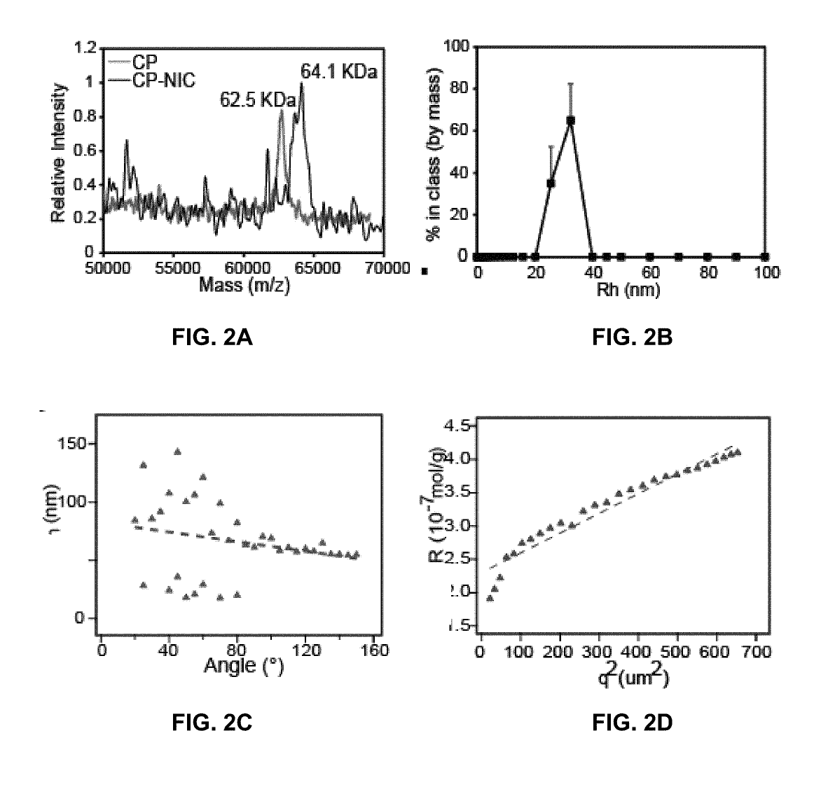

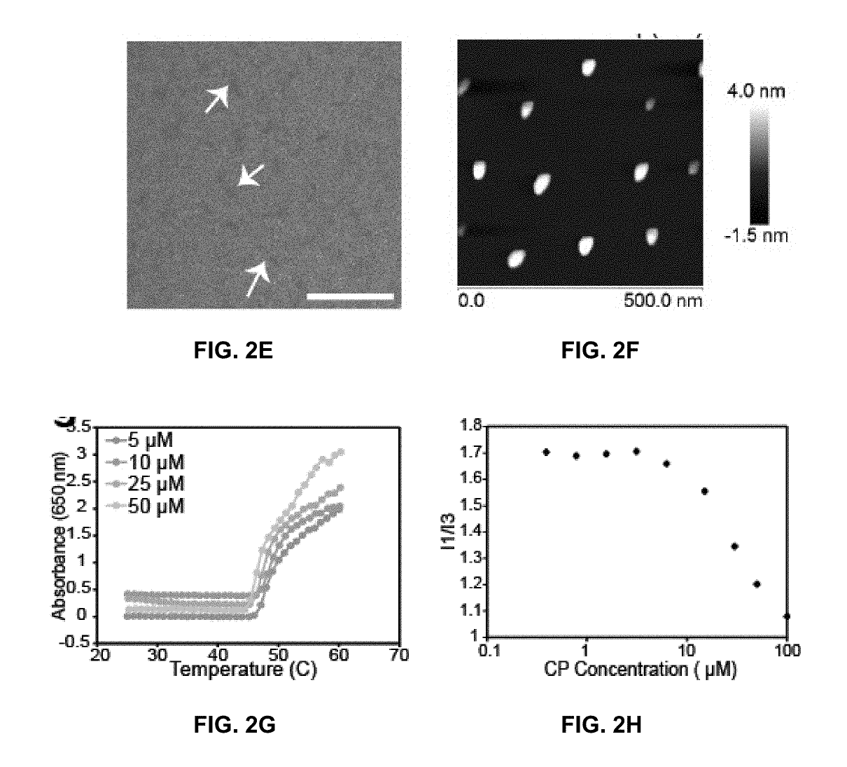

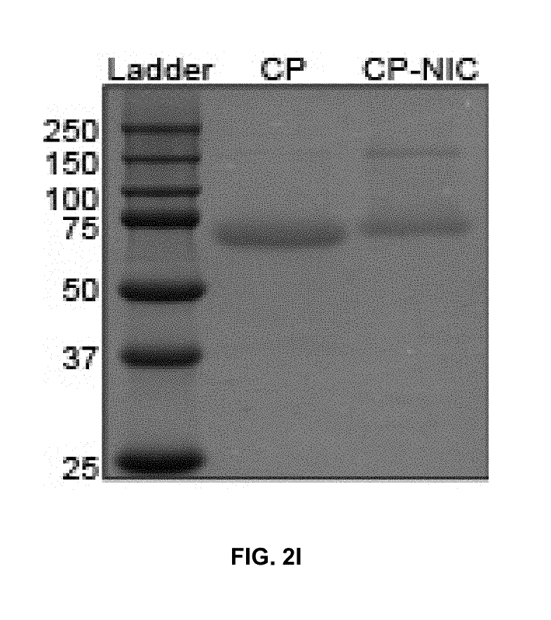

Matrix-assisted laser desorption/ionization time-of-flight mass spectrometry (MALDI-TOF MS) and SDS-PAGE analysis (FIGS. 2A and 2I, respectively) showed that the molecular weight of the CP is 62550 Da. Purified CP—NIC conjugate has ˜4 drug molecules per CP, as determined from the mass difference between the conjugate and the parent CP measured by MALDI-TOF MS (FIG. 2A). The drug loading efficiency of 2 wt. % of the conjugate was consistent with previous CP-drug conjugates (MacKay et al., Nat. Mater, 2009, 8, 993-999; Bhattacharyya et al., Nat. Commun., 2015, 6, 7939). In principle, up to 7 drug molecules can be conjugated per CP molecule. The 2 wt. % loading suggests an average of 4 NIC molecules per CP. The CP—NIC conjugate did not ionize well in MALDI, and the [M+H]⁺ peak for the CP—NIC conjugate had relatively low intensity and low signal-to-noise ratio. As a result, it was difficult to determine the poly-dispersity of CP—NIC from the peak width by deconvolution of the peak into components representing CP—NIC conjugates with different stoichiometry.

Some of the physicochernical properties of a representative CP—NIC conjugate is shown in Table 1.

TABLE 1

| CP-polypeptide sequence | SKGPG(XGVPG)₁₆₀WPC(GGC)₇ |
|---|---|
| Guest residues (X) | V:A:G [1:8:7] |
| Molecular weight of CP (KDa) | 62.5 |
| ¹Drugs per CP | 4 |
| ²Rₕ (nm) | 30.1 ± 10.4 |
| ²R_{g} (nm) | 81.5 ± 5.8% |
| ³Z (chains per nanoparticle) | 90 |
| ρ | 1.65 |
| MW (g/mol) | 5.83 × 10⁶ ± 3.9% |
| CMC (μM) | 3.1 |

¹Drug molecules calculated from MALDI MS.
²Rₕ determined by DLS at 25° C. in PBS. Mean ± % PD (n = 3).
³Aggregation number (Z): Number of CP-NIC molecules per nanoparticle, as determined by SLS.

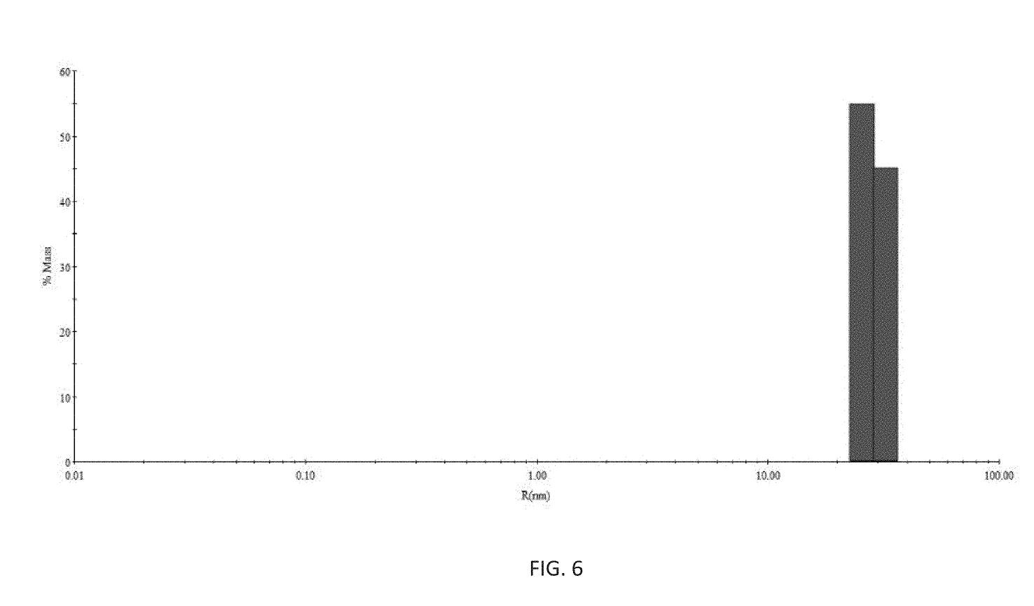

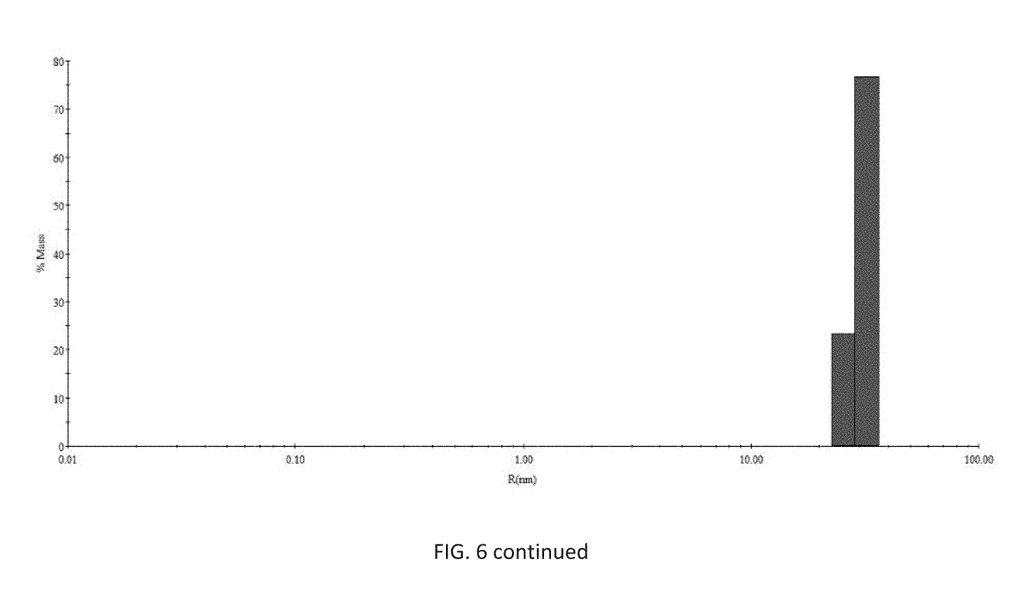

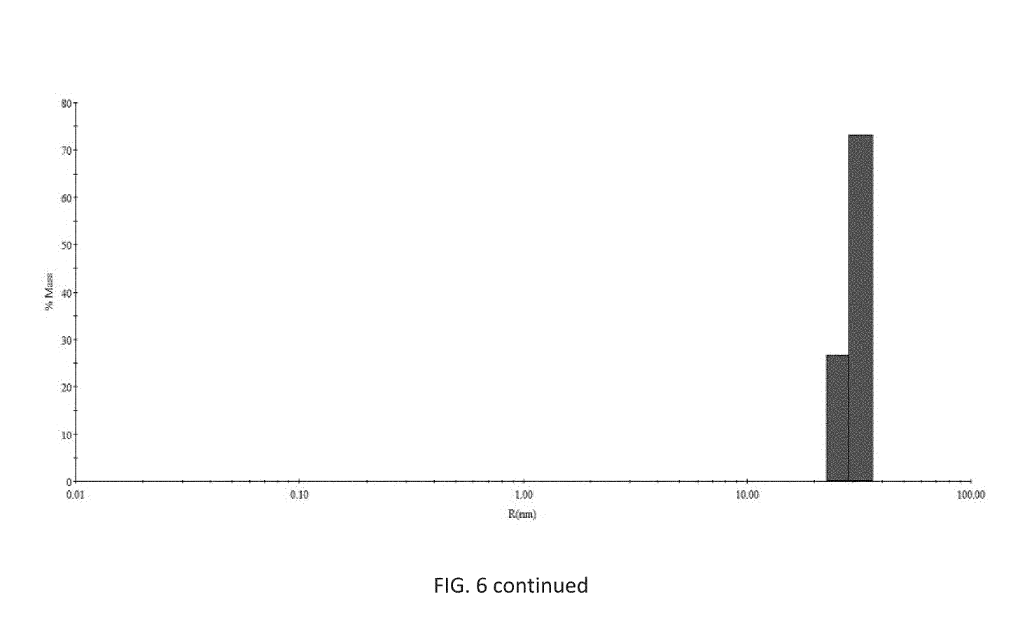

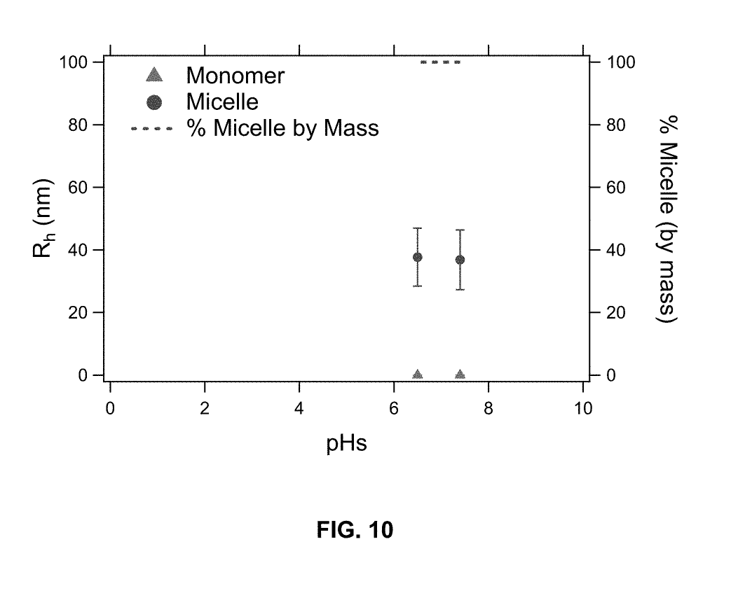

Upon conjugation with NIC, the CP—NIC conjugate spontaneously self-assembled into near-monodisperse cylindrical micelles (FIG. 1B). As NIC is a hydrophobic drug with a log D of 4.48 at pH 7, these results are consistent with our previous observation that conjugation of multiple copies of a hydrophobic small molecule drug with a log D>1.5 to one end of a hydrophilic polypeptide (CP) impart sufficient amphiphilicity to trigger the self-assembly of CP into nanoparticles. The radius of gyration (R_{g}), and hydrodynamic radius (Rₕ) of CP—NIC conjugate were determined by static and dynamic light scattering (SLS/DLS). The Rₕ of CP—NIC conjuagte measured by fixed angle DLS using a DynaPro™ Plate Reader (Wyatt Technology; Santa Barbara, Calif.) at 25° C. To obtain histograms, regularization fits were used to determine the hydrodynamic radius as weighted by the percent by mass and the Rh of CP—NIC conjugate was calculated as 30 nm (FIG. 2B, FIG. 6, and FIG. 10). However, the Rₕ calculated from the SLS/DLS measurement with ALV instrument was 49.3 nm (Table 1). Without being limited to any particular theory, it was hypothesized that such discrepancy was likely due to the fact that the SLS and DLS measurement on the ALV instrument are carried out simultaneously in the angular range of 30°-150° at 5° increments, and the Rₕ was calculated from the inflection point at 00, whereas the DLS measurement in the Wyatt instrument is at a fixed angle of 145°. Analysis of the partial Zimm plot obtained from SLS showed that the R, of the CP—NIC nanoparticles was 81.5 nm, and that the aggregation number of the nanoparticles was 90 (FIGS. 2B-2C and Table 1). The experimentally determined form factor (p)-calculated as R_{g}/Rₕ-was 1.65, which is close to the theoretical value for cylindrical particles with high aspect ratio.

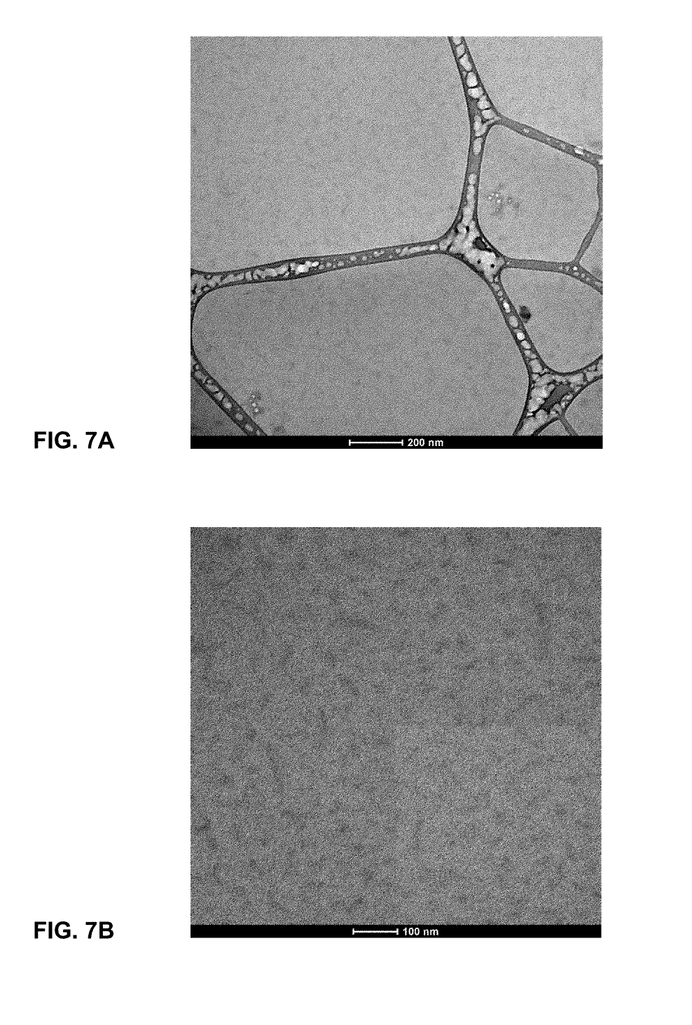

The size and rod-like morphology of the CP—NIC nanoparticles were confirmed by cryo-TEM, which allows for the direct visualization of self-assembled structures in a near-native, hydrated state (FIG. 2E and FIG. 7). Only the hydrophobic core of CP—NIC nanoparticles was visualized by cryo-TEM, due to the low electron density and high degree of hydration of the ELP chains in the corona of the nanoparticles. However, it was difficult to gain better contrast in cryo-TEM in these experiments. The contrast in cryo-TEM was generated from differences in electron density between the sample and the vitreous ice layer. The contrast for the tested polypeptide materials in cryo-TEM was limited by two factors. The polypeptides are composed of relatively light atoms and have lower electron density than many synthetic polymers, and thus exhibit low contrast. Furthermore, the hydrophobic cores of the assembled nanoparticles likely remained hydrated, further limiting the difference in electron density between the core and the solvent. This is in contrast to synthetic diblock copolymers where the core forming domain is typically much less hydrated, and hence provides good contrast in cryo-TEM. Without being limited to any particular theory, it was hypothesized that the core's low electron density and its likely high degree of hydration in combination limited the contrast achievable by cryo-TEM.

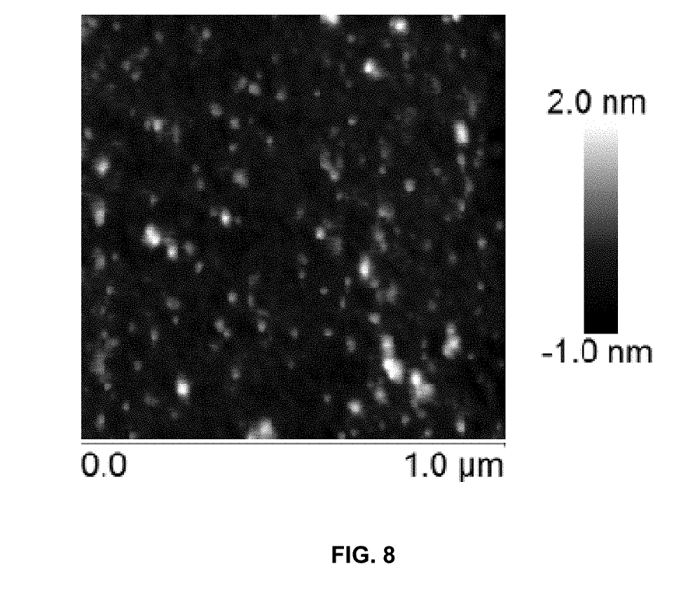

It is possible to obtain greater contrast, and thus more easily interpretable images, through negative staining and conventional TEM, but this comes at the cost of potential major changes to the sample size and morphology during the sample preparation process. It was hypothesized that despite the low contrast images, cryo-TEM, which captures images of the micelles in their near-native state, combined with light scattering may be a preferred approach to characterize polypeptide self-assembly. The average length of the cylindrical nanoparticle determined by cryo-TEM (LTEM) was measured as 74±10 nm (n=10), and the average diameter (DTEM) was measured as 12.5±3.5 nm. The micellar morphologies were further verified by atomic force microscopy (AFM) under ambient condition (FIG. 2F and FIG. 8). The AFM images show distinct particles with a rod or worm-like morphology. The observed width of the worm-like micelle was much larger than their heights, which was likely attributed to the spreading of the micelles on the mica surface during sample preparation and also because of the tip-induced broadening effect inherent to AFM.

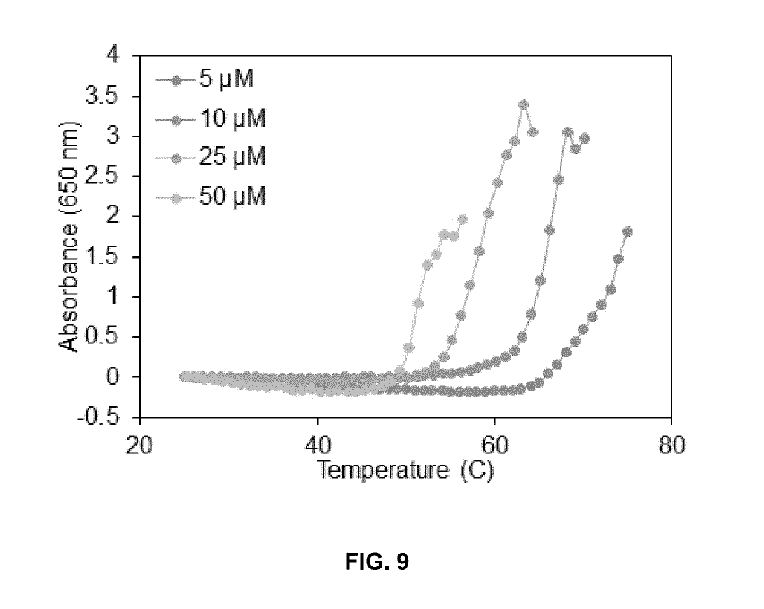

CPs are thermally responsive and display lower critical solution temperature (LCST) phase transition behavior, in which the protein goes from a soluble state to an insoluble coacervate phase upon raising the solution temperature above its cloud point, also called the inverse transition temperature (Tt). The thermal responsiveness of the CP—NIC nanoparticles was measured here as a function of the CP concentration in mouse serum to model the physiological milieu that the CP—NIC nanoparticles would be in upon i.v. injection (FIG. 2G). In serum, the Tt of the CP—NIC nanoparticles was independent of the CP—NIC concentration in the range of 5-50 μM (45° C. at 25 μM), which is in sharp contrast to unconjugated CP, where Tt varied significantly with concentration (ranging from 48° C. for 50 μm to 65° C. for 5 μM) (FIG. 9). This result is consistent with previous studies of other drug conjugates that form nanoparticles (MacKay et al., Nat. Mater., 2009, 8, 993-999; Bhattacharyya et al., Nat. Commun., 2015, 6, 7939), and suggests that the high local polypeptide concentration in the CP—NIC nanoparticles makes the Tt nearly independent of its overall, solution concentration.

CP—NIC nanoparticles were further characterized by fluorescence spectroscopy using pyrene as a probe of local hydrophobicity, which enables measurement of the critical aggregation concentration (CAC) of the self-assembled nanoparticles. The ratio of the first fluorescence emission peak (I₃₇₀₋₃₇₃) to the third peak (I₃₂₁₋₃₈₄) was plotted over a range of CP concentrations (FIG. 2F). The sigmoid of best fit was used to calculate the CAC, defined as the inflection point of the curve, giving the CAC of the CP—NIC nanoparticles of -3 μM (FIG. 2F).

### Example 4. In Vitro Anti-Cancer Efficacy

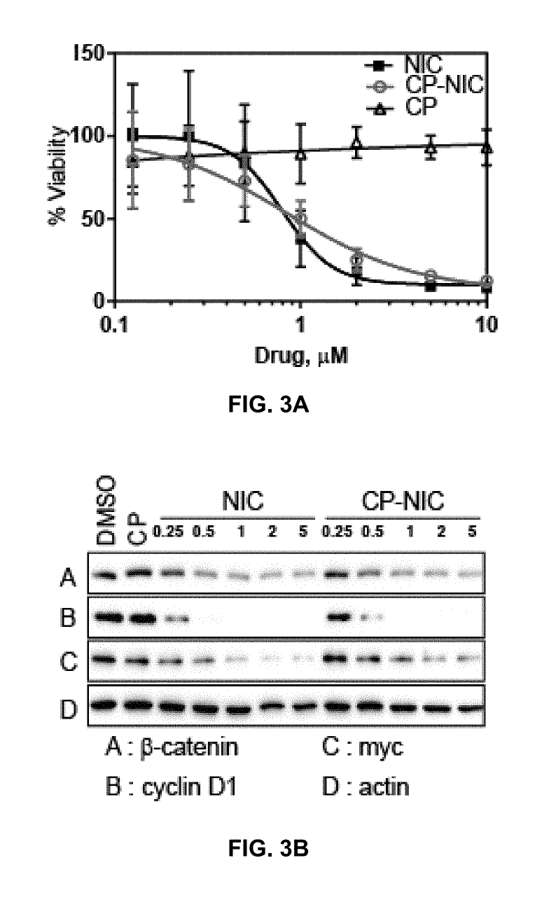

Based on the observation that NIC was packaged in the core of a representative CP—NIC nanoparticle, further studies were conducted to verify that such formulation may retain the therapeutic activity of the NIC compound. Prior SAR studies indicated that the ester attachment did not affect NIC activity (Mook et al., Bioorg. Med. Chem. Lett., 2015, 23, 5829-5838). Human colon carcinoma HCT116 cell line was used to evaluate the in vitro cytotoxicity of the CP—NIC conjugate, as NIC has been proposed for clinical use in human colon carcinoma. After 72 hours of exposure to CP—NIC nanoparticles, HCT116 cell proliferation was significantly inhibited (FIG. 3A). The IC₅₀, defined as the concentration of NIC (or NIC equivalent for the CP—NIC nanoparticles) needed to inhibit the proliferation of cells by 50%, was found to be 0.94 μM for CP—NIC and 0.85 μM for free NIC.

The efficacy of CP—NIC to inhibit Wnt signaling in HCT116 cells was also determined. Wnt signaling activity was quantified as cytosolic β-catenin level by Western blot. Upon treatment of HCT116 cells with CP—NIC at doses ranging from 0.25 to 5 μM (NIC equivalent) for 18 hours, β-catenin levels were significantly decreased in HCT116 cells (FIG. 3B), similar to the inhibition observed for the same equivalent dose of free NIC. Levels of the Wnt target proteins c-myc and cyclin D1 were also similarly reduced by treatment with NIC, as shown by western blotting. These data, together with data from the cell proliferation assay, clearly demonstrate that the CP—NIC nanoparticles inhibit the in vitro proliferation of HCT116 cells and the Wnt signaling pathway, and that conjugation of NIC to CP does not significantly decrease the activity of the drug.

### Example 5. Pharmacokinetic Analysis of CP—NIC

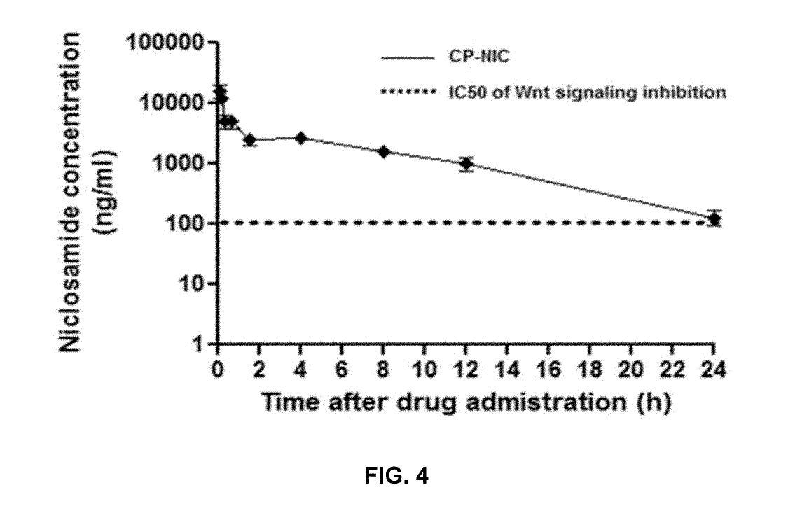

To compare the plasma exposure to NIC from CP—NIC nanoparticles versus NIC, CP—NIC nanoparticles or NIC were administered intravenously and the plasma NIC concentration was measured as a function of time post-injection (FIG. 4). LCMS-MS analysis was employed to determine the in vivo concentration of NIC as free drug liberated from CP—NIC nanoparticles. The mechanism of cleavage of NIC from the particle has not been defined, however it was hypothesized that such mechanism may involve protease cleavage and/or aqueous hydrolysis. The pharmacokinetic parameters were calculated using a non-compartment pharmacokinetic approach using the WinNonlin software, yielding a terminal half-life of NIC derived from the CP—NIC nanoparticles of 4.2±1.34 h and a plasma AUC of 36.9±7.34 μg/mL/h. In contrast, the terminal half-life and AUC for NIC in mice (treated at the same dose of unconjugated NIC) are only 1.0±0.22 h and 3.3±1.3 μg/mL/h respectively. Table 2 shows the pharmacokinetic parameters of NIC delivered by a representative CP—NIC nanoparticles, and those of the free drug.

TABLE 2

| PK parameter | NIC | CP-NIC |
|---|---|---|
| Cₘₐₓ, mg/mL | 6.2 ± 4.23 | 28.7 ± 32.16 |
| tₘₐₓ, h | 0.2 ± 0.17 | 0.04 ± 0.08 |
| AUCₗₐₛₜ (area under curve, up to last | 3.3 ± 1.30 | 36.9 ± 7.34 |
| measured point), h * mg/mL | | |
| AUC_{inf} (extrap. to infinity), h * mg/mL | 3.3 ± 1.31 | 37.7 ± 7.73 |
| % AUC-inf extrapolated | 0.33 ± 0.45 | 2.1 ± 1.33 |
| ((AUC-last/AUC-inf) * 100 | | |
| t_{1/2} (half-life of the terminal process), h | 1.0 ± 0.22 | 4.2 ± 1.34 |
| CL (clearance = dose/AUC-inf), L/h | 0.89 ± 0.45 | 0.07 ± 0.01 |
| (per kg BW) | | |
| MRTₗₐₛₜ (mean residence in body, | 0.9 ± 0.44 | 5.8 ± 0.95 |
| up to last measured point), h | | |
| MRT_{inf} (mean residence in body, | 0.9 ± 0.48 | 6.4 ± 0.93 |
| extrapolated), h | | |
| Vₛₛ (=CL × MRT; overall distrib. | 0.7 ± 0.28 | 0.5 ± 0.12 |
| Volume at steay state), L (per kg BW) | | |

In fact, the plasma levels of NIC obtained by dosing CP—NIC at 128 mg CP—NIC Equiv/kg BW remained above the IC₅₀ of inhibition of Wnt signaling by NIC in the TOPFlash assay for nearly 24 h (FIG. 4), whereas the reported plasma levels of NIC dosed as a free drug solution at 200 mg/kg BW were only above the IC₅₀ for Wnt inhibition for less than 1 h (Osada et al., Cancer Res., 2011, 71, 4172-4182).

### Example 6. In Vivo Anti-Tumor Activity

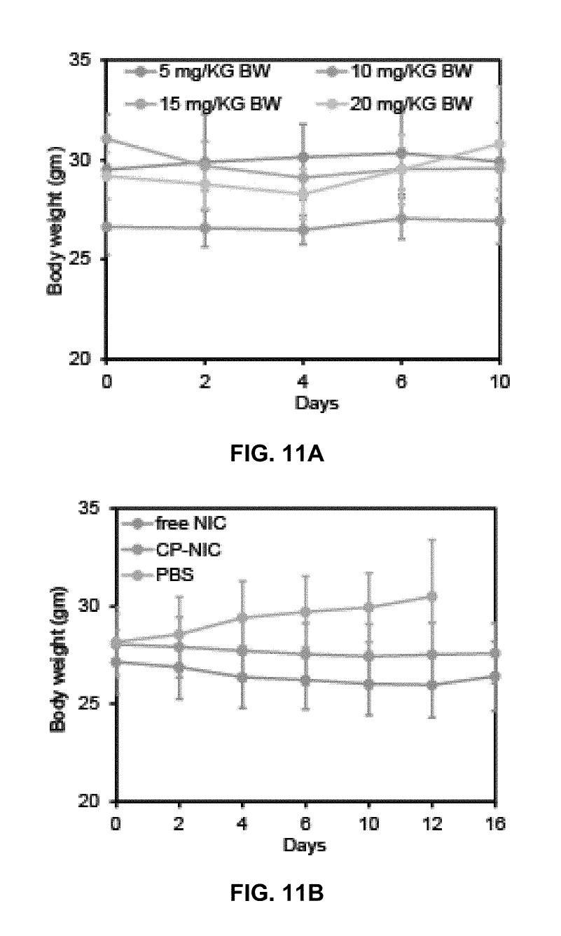

To compare the therapeutic effect of CP—NIC nanoparticles versus free NIC, CP—NIC formulations were administered in a dose escalation study. The maximum deliverable dose (MDD) of CP—NIC due to solution viscosity was 20 mg NIC Equiv/kg BW (FIG. 11). It was hypothesized that the maximum tolerated dose (MTD) of CP—NIC nanoparticles may be greater than 20 mg/kg, however higher doses beyond this level were not tested due to viscosity of the solution.

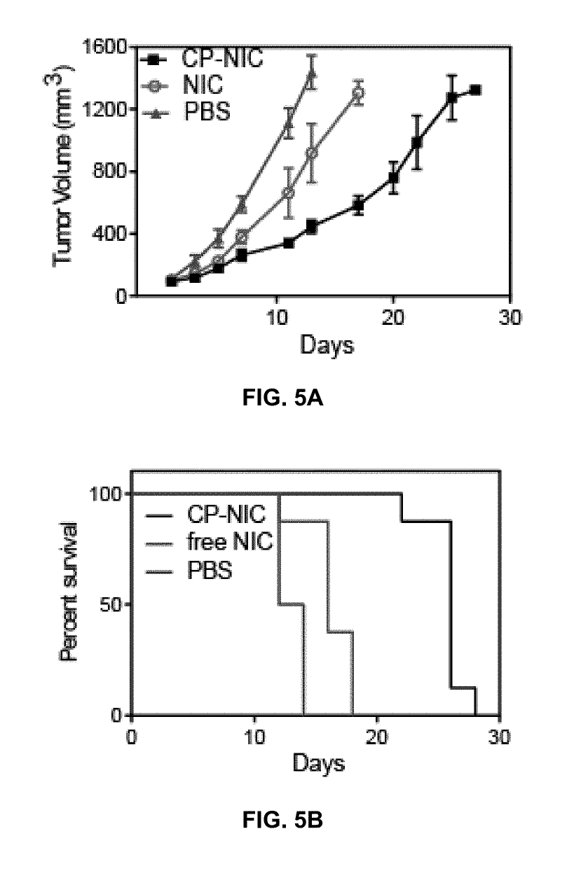

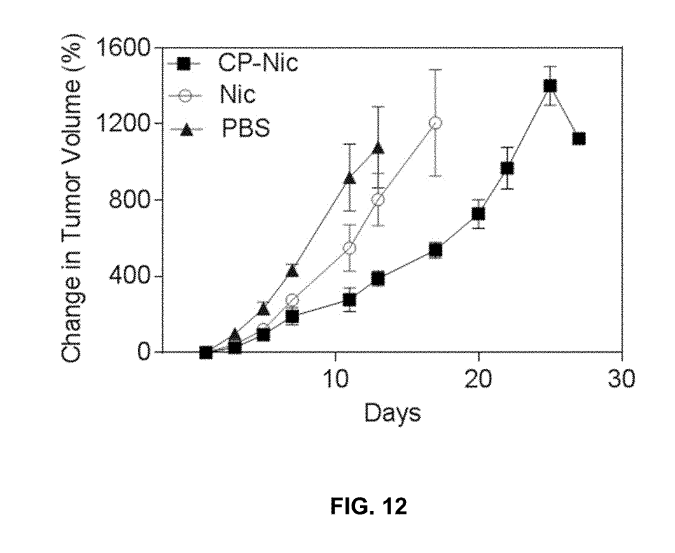

Next, the tumor inhibition efficacy of the maximum deliverable/tolerated dose of CP—NIC versus free NIC was evaluated in the HCT-116 cell xenograft model. Mice with HCT-116 tumors were treated every third day for two weeks intravenously with PBS, unconjugated NIC (5 mg/kg), or CP—NIC nanoparticles (20 mg/kg of NIC-equivalent) (FIG. 5A). The 5 mg/kg dose of free NIC was chosen because in a pilot study with NIC formulated in a mixture of N-dimethylacetamide (DMA) and Polyethylene Glycol 400 (1:2 v/v), the LD₅₀ of NIC in nude mice was found to be 5 mg/kg BW (data not included). Body-weight loss was also measured throughout the treatment of free NIC and CP—NIC conjugate. All treatments were tolerated for the period of the study (FIG. 11). Ten days after the start of the treatment, CP—NIC treated mice had a mean tumor volume of 339 mm3 (n=8) versus 661 mm3 (n=8) for NIC-treated (Tukey; p=0.001), compared to 1111 mm3 (n=8) for PBS-treated controls (Tukey; p=0.0001). The CP—NIC formulation outperforms free drug in reducing growth in tumor volume, which correlated with extended animal survival (FIG. 5B and FIG. 12). The median survival time for mice treated with PBS (n=8) was 13 days, and treatment with the free NIC (n=8) slightly increased survival to 16 days (Kaplan-Meier, log-rank test, p<0.0001). Treatment with CP—NIC (n=8) further increased survival to 26 days (Kaplan-Meier, log-rank test, p<0.0001). It was hypothesized that the mechanism of action of NIC released from nanoparticles in this study is the same as free NIC as previously characterized. These results demonstrate that treatment with CP—NIC nanoparticles improve the survival of mice bearing a subcutaneous HCT-116 cell tumor, compared to treatment with free NIC drug.

The breadth and scope of the present disclosure should not be limited by any of the above-described exemplary aspects. Various features and advantages of the invention are set forth in the following claims.

For reasons of completeness, various aspects of the invention are set out in the following numbered clauses:

Clause 1. A conjugate of formula (I), or a pharmaceutically acceptable salt thereof,

Z-(-L-D)ₚ (I)

wherein,

Z is a polypeptide having a cysteine-enriched segment;

p is 1 to 8;

each -L-D group is covalently attached to the cysteine-enriched segment;

L is linker;

D is

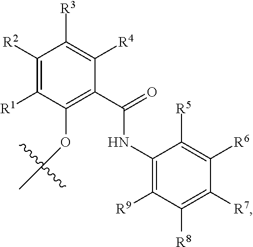

wherein
R¹, R², R³, R⁴, R⁵, R⁶, R⁸, and R⁹ at each occurrence are independently hydrogen, halogen, C₁-C₆ alkyl, C₁-C₆ haloalkyl, or C₁-C₆ alkoxy; and
R⁷ at each occurrence is halogen, C₁-C₆ haloalkyl, —NO₂, or —SO₂—C₁-C₄ alkyl.

Clause 2. The conjugate of clause 1, or a pharmaceutically acceptable salt thereof, wherein the polypeptide is an elastin-like polypeptide (ELP).

Clause 3. The conjugate of clause 2, or a pharmaceutically acceptable salt thereof, wherein the cysteine-enriched segment is at the C-terminus of the polypeptide.

Clause 4. The conjugate of clause 1, or a pharmaceutically acceptable salt thereof, wherein the cysteine-enriched segment comprises (Gly-Gly-Cys)ₙ, wherein n is 2 to 10.

Clause 5. The conjugate of clause 1, or a pharmaceutically acceptable salt thereof, wherein L is

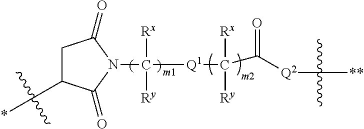

wherein

Q¹ is bond, —O—, —NH—, aryl, cycloalkyl, heterocyclyl, heteroaryl, —(CH₂CH₂O)ₘ₃—, wherein the aryl, cycloalkyl, heterocyclyl, and heteroaryl are each optionally substituted by at least one C₁-C₄ alkyl, halogen, or C₁-C₄ haloalkyl;

Q² is bond, —O—CH(R^{w})—, -AAᵣ-, —Yₜ—, or -AAᵣ-Yₜ—, in which Y, if present, is attached to D;

R^{w} is H, C₁-C₆ alkyl, aryl, or cycloalkyl;

R^{x} and R^{y} at each occurrence are independently hydrogen or C₁-C₄ alkyl;

AA at each occurrence is independently an amino acid unit;

Y at each occurrence is independently a self-immolative spacer unit;

m1 is 0 to 10, provided that when Q1 is bond, m1 is 1-10;

m2 is 0 to 10;

m3 is 0 to 20;

r is 1-10;

t is 1 or 2;

* indicates attachment to Z;

** indicates attachment to D.

Clause 6. The conjugate of clause 5, or a pharmaceutically acceptable salt thereof, wherein Q² is bond.

Clause 7. The conjugate of clause 6, or a pharmaceutically acceptable salt thereof, wherein Q¹ is bond, and m2 is 0.

Clause 8. The conjugate of clause 5, or a pharmaceutically acceptable salt thereof, wherein Q² is -AAᵣ-.

Clause 9. The conjugate of clause 5, or a pharmaceutically acceptable salt thereof, wherein L is

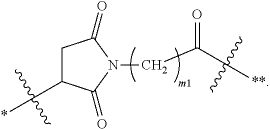

Clause 10. The conjugate of clause 5, or a pharmaceutically acceptable salt thereof, wherein Q¹ is cycloakyl.

Clause 11. The conjugate of clause 10, or a pharmaceutically acceptable salt thereof, wherein L is

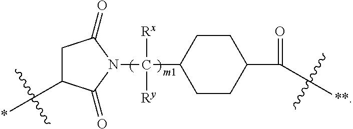

Clause 12. The conjugate of clause 1, or a pharmaceutically acceptable salt thereof, where -L-D is

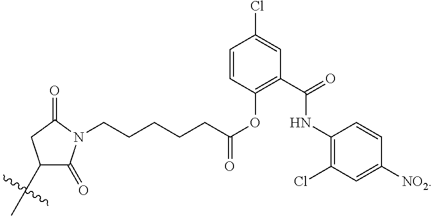 [SMILES: *C1CC(=O)N(CCCCCC(=O)Oc2ccc(Cl)cc2C(=O)Nc2ccc([N+](=O)[O-])cc2Cl)C1=O]

Clause 13. The conjugate of clause 1, or a pharmaceutically acceptable salt thereof, wherein p is 1, 2, 3, or 4.

Clause 14. A nanoparticle comprising the conjugate of clause 1 or a pharmaceutically acceptable salt thereof, wherein the -D groups of the conjugate form a core of the nanoparticle.

Clause 15. A pharmaceutical composition comprising the conjugate of clause 1 or a pharmaceutically acceptable salt thereof and a pharmaceutically acceptable carrier.

Clause 16. A compound of formula (II), or a pharmaceutically acceptable salt thereof,

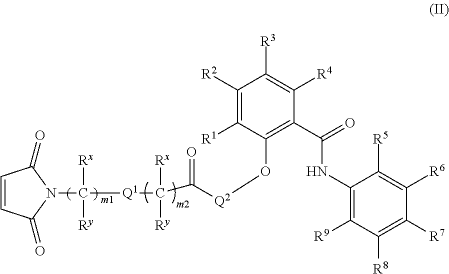

wherein,

Q¹ is bond, —O—, —NH—, aryl, cycloalkyl, heterocyclyl, heteroaryl, —(CH₂CH₂O)ₘ₃—, wherein the aryl, cycloalkyl, heterocyclyl, and heteroaryl are each optionally substituted by at least one C₁-C₄ alkyl, halogen, or C₁-C₄ haloalkyl;

Q² is bond, —O—CH(R^{w})—, -AAᵣ-, —Yₜ—, or -AAᵣ-Yₜ—, in which Y, if present, is attached to the

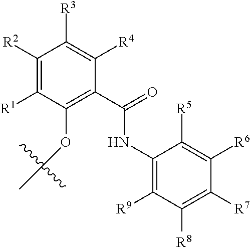

group;

R^{w} is H, C₁-C₆ alkyl, aryl, or cycloalkyl;

R^{x} and R^{y} at each occurrence are independently hydrogen or C₁-C₄ alkyl;

AA at each occurrence is independently an amino acid unit;

Y at each occurrence is independently a self-immolative spacer unit;

m1 is 0 to 10, provided that when Q¹ is bond, m1 is 1-10;

m2 is 0 to 10;

m3 is 0 to 20;

r is 1-10;

t is 1 or 2;

R¹, R², R³, R⁴, R⁵, R⁶, R⁸, and R⁹ at each occurrence are independently hydrogen, halogen, C₁-C₆ alkyl, C₁-C₆ haloalkyl, or C₁-C₆ alkoxy; and

R⁷ at each occurrence is halogen, C₁-C₆ haloalkyl, —NO₂, or —SO₂—C₁-C₄ alkyl.

Clause 17. The compound of clause 16, or a pharmaceutically acceptable salt thereof, wherein Q² is bond.

Clause 18. The compound of clause 16, or a pharmaceutically acceptable salt thereof, wherein the compound has a structure of formula (II-a)

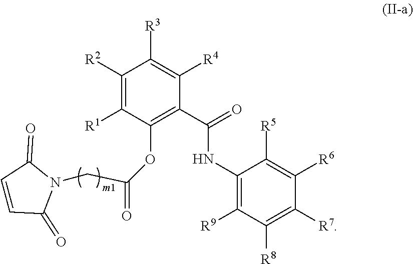

Clause 19. The compound of clause 16, or a pharmaceutically acceptable salt thereof, wherein the compound has a structure of formula (II-b)

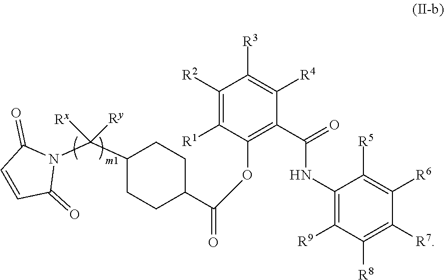

Clause 20. The compound of clause 16, or a pharmaceutically acceptable salt thereof, wherein the compound is

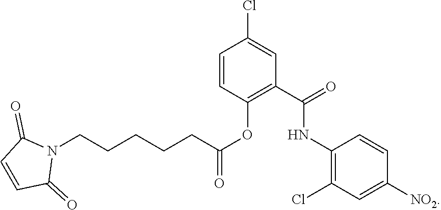 [SMILES: O=C(CCCCCN1C(=O)C=CC1=O)Oc1ccc(Cl)cc1C(=O)Nc1ccc([N+](=O)[O-])cc1Cl]

Clause 21. A method of preparing a conjugate or a pharmaceutically acceptable salt thereof, comprising the steps of:

preparing a polypeptide having a cysteine-enriched segment; and

reacting the polypeptide with a compound of clause 16 or a pharmaceutically acceptable salt thereof to form the conjugate, wherein the compound is covalently attached to the cysteine-enriched segment of the polypeptide.

Clause 22. The method of clause 21, wherein the polypeptide is an elastin-like polypeptide (ELP).

Clause 23. The method of clause 22, wherein the cysteine-enriched segment is at the C-terminus of the polypeptide.

Clause 24. The method of clause 23, wherein the cysteine-enriched segment comprises (Gly-Gly-Cys)ₙ, wherein n is 2 to 10.

Clause 25. A method for treating a disease in a subject in need thereof, comprising administering to a subject in need thereof an effective amount of a pharmaceutical composition comprising the conjugate of clause 1 or a pharmaceutically acceptable salt thereof and a pharmaceutically acceptable carrier.

Clause 26. The method of clause 25, wherein the disease is cancer, parasite infection, bacterial infection, viral infection, metabolic diseases, Type II diabetes, NASH, NAFLD, artery constriction, endometriosis, neuropathic pain, rheumatoid arthritis, sclerodermatous graft-versus-host disease, systemic sclerosis, or combinations thereof.

Clause 27. The method of clause 26, wherein the polypeptide is an elastin-like polypeptide (ELP) and the cysteine-enriched segment is at the C-terminus of the polypeptide.

Clause 28. The method of clause 27, wherein the cysteine-enriched segment comprises (Gly-Gly-Cys)ₙ, wherein n is 2 to 10.

Clause 29. The method of clause 26, wherein the -L-D group of the conjugate or a pharmaceutically acceptable salt thereof has a structure of

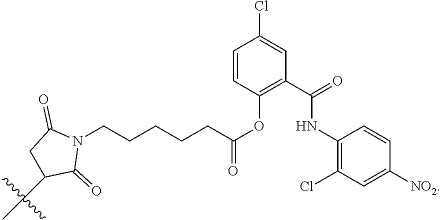 [SMILES: *C1CC(=O)N(CCCCCC(=O)Oc2ccc(Cl)cc2C(=O)Nc2ccc([N+](=O)[O-])cc2Cl)C1=O]

Clause 30. The method of clause 26, wherein p is 1, 2, 3, or 4.

1. A conjugate of formula (I), or a pharmaceutically acceptable salt thereof,
Z-(-L-D)ₚ (I)
wherein,
Z is a polypeptide having a cysteine-enriched segment;
p is 1 to 8;
each -L-D group is covalently attached to the cysteine-enriched segment;
L is linker;
D is 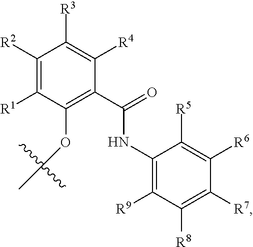
wherein
R¹, R², R³, R⁴, R⁵, R⁶, R⁸, and R⁹ at each occurrence are independently hydrogen, halogen, C₁-C₆ alkyl, C₁-C₆ haloalkyl, or C₁-C₆ alkoxy; and
R⁷ at each occurrence is halogen, C₁-C₆ haloalkyl, —NO₂, or —SO₂—C₁-C₄ alkyl.

2. The conjugate of claim 1, or a pharmaceutically acceptable salt thereof, wherein the polypeptide is an elastin-like polypeptide (ELP).

3. The conjugate of claim 2, or a pharmaceutically acceptable salt thereof, wherein the cysteine-enriched segment is at the C-terminus of the polypeptide.

4. The conjugate of claim 1, or a pharmaceutically acceptable salt thereof, wherein the cysteine-enriched segment comprises (Gly-Gly-Cys)ₙ, wherein n is 2 to 10.

5. The conjugate of claim 1, or a pharmaceutically acceptable salt thereof, wherein L is 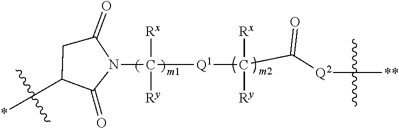
wherein
Q¹ is bond, —O—, —NH—, aryl, cycloalkyl, heterocyclyl, heteroaryl, —(CH₂CH₂O)ₘ₃—, wherein the aryl, cycloalkyl, heterocyclyl, and heteroaryl are each optionally substituted by at least one C₁-C₄ alkyl, halogen, or C₁-C₄ haloalkyl;
Q² is bond, —O—CH(R^{w})—, -AAᵣ-, —Yₜ—, or -AAᵣ-Yₜ—, in which Y, if present, is attached to D;
R^{w} is H, C₁-C₆ alkyl, aryl, or cycloalkyl;
R^{x} and R^{y} at each occurrence are independently hydrogen or C₁-C₄ alkyl;
AA at each occurrence is independently an amino acid unit;
Y at each occurrence is independently a self-immolative spacer unit;
m1 is 0 to 10, provided that when Q¹ is bond, m1 is 1-10;
m2 is 0 to 10;
m3 is 0 to 20;
r is 1-10;
t is 1 or 2;
* indicates attachment to Z;
** indicates attachment to D.

6. The conjugate of claim 5, or a pharmaceutically acceptable salt thereof, wherein Q² is bond.

7. The conjugate of claim 6, or a pharmaceutically acceptable salt thereof, wherein Q¹ is bond, and m2 is 0.

8. The conjugate of claim 5, or a pharmaceutically acceptable salt thereof, wherein Q² is -AAᵣ-.

9. The conjugate of claim 5, or a pharmaceutically acceptable salt thereof, wherein L is

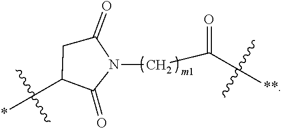

10. The conjugate of claim 5, or a pharmaceutically acceptable salt thereof, wherein Q¹ is cycloakyl.

11. The conjugate of claim 10, or a pharmaceutically acceptable salt thereof, wherein L is

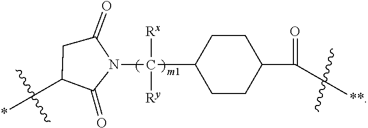

12. The conjugate of claim 1, or a pharmaceutically acceptable salt thereof, where -L-D is

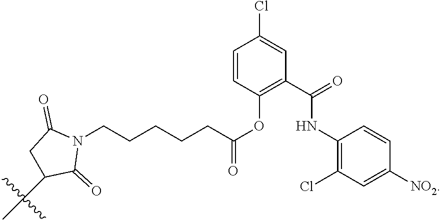 [SMILES: *C1CC(=O)N(CCCCCC(=O)Oc2ccc(Cl)cc2C(=O)Nc2ccc([N+](=O)[O-])cc2Cl)C1=O]

13. The conjugate of claim 1, or a pharmaceutically acceptable salt thereof, wherein p is 1, 2, 3, or 4.

14. A nanoparticle comprising the conjugate of claim 1 or a pharmaceutically acceptable salt thereof, wherein the -D groups of the conjugate form a core of the nanoparticle.

15. A pharmaceutical composition comprising the conjugate of claim 1 or a pharmaceutically acceptable salt thereof and a pharmaceutically acceptable carrier.

16. A compound of formula (II), or a pharmaceutically acceptable salt thereof, 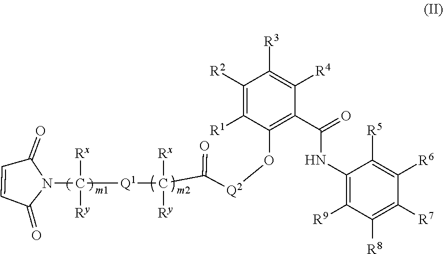
wherein,
Q¹ is bond, —O—, —NH—, aryl, cycloalkyl, heterocyclyl, heteroaryl, —(CH₂CH₂O)ₘ₃—, wherein the aryl, cycloalkyl, heterocyclyl, and heteroaryl are each optionally substituted by at least one C₁-C₄ alkyl, halogen, or C₁-C₄ haloalkyl;
Q² is bond, —O—CH(R^{w})—, -AAᵣ-, —Yₜ—, or -AAᵣ-Yₜ—, in which Y, if present, is attached to the 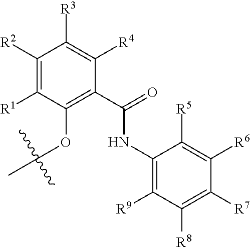
group;
R^{w} is H, C₁-C₆ alkyl, aryl, or cycloalkyl;
R^{x} and R^{y} at each occurrence are independently hydrogen or C₁-C₄ alkyl;
AA at each occurrence is independently an amino acid unit;
Y at each occurrence is independently a self-immolative spacer unit;
m1 is 0 to 10, provided that when Q¹ is bond, m1 is 1-10;
m2 is 0 to 10;
m3 is 0 to 20;
r is 1-10;
t is 1 or 2;
R¹, R², R³, R⁴, R⁵, R⁶, R⁸, and R⁹ at each occurrence are independently hydrogen, halogen, C₁-C₆ alkyl, C₁-C₆ haloalkyl, or C₁-C₆ alkoxy; and
R⁷ at each occurrence is halogen, C₁-C₆ haloalkyl, —NO₂, or —SO₂—C₁-C₄ alkyl.

17. The compound of claim 16, or a pharmaceutically acceptable salt thereof, wherein Q² is bond.

18. The compound of claim 16, or a pharmaceutically acceptable salt thereof, wherein the compound has a structure of formula (II-a)

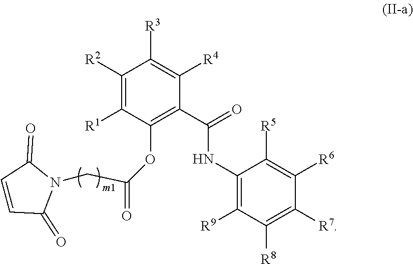

19. The compound of claim 16, or a pharmaceutically acceptable salt thereof, wherein the compound has a structure of formula (II-b)

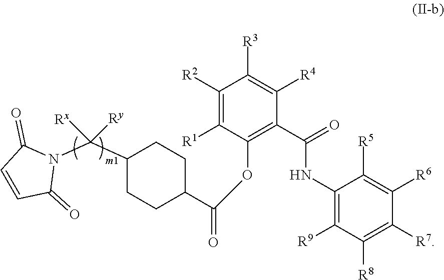

20. The compound of claim 16, or a pharmaceutically acceptable salt thereof, wherein the compound is

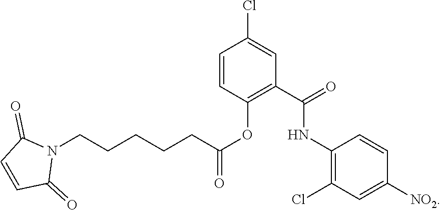 [SMILES: O=C(CCCCCN1C(=O)C=CC1=O)Oc1ccc(Cl)cc1C(=O)Nc1ccc([N+](=O)[O-])cc1Cl]

21. A method of preparing a conjugate or a pharmaceutically acceptable salt thereof, comprising the steps of:
preparing a polypeptide having a cysteine-enriched segment; and
reacting the polypeptide with a compound of claim 16 or a pharmaceutically acceptable salt thereof to form the conjugate, wherein the compound is covalently attached to the cysteine-enriched segment of the polypeptide.

22. The method of claim 21, wherein the polypeptide is an elastin-like polypeptide (ELP).

23. The method of claim 22, wherein the cysteine-enriched segment is at the C-terminus of the polypeptide.

24. The method of claim 23, wherein the cysteine-enriched segment comprises (Gly-Gly-Cys)ₙ, wherein n is 2 to 10.

25. A method for treating a disease in a subject in need thereof, comprising administering to a subject in need thereof an effective amount of a pharmaceutical composition comprising the conjugate of claim 1 or a pharmaceutically acceptable salt thereof and a pharmaceutically acceptable carrier.

26. The method of claim 25, wherein the disease is cancer, parasite infection, bacterial infection, viral infection, metabolic diseases, Type II diabetes, NASH, NAFLD, artery constriction, endometriosis, neuropathic pain, rheumatoid arthritis, sclerodermatous graft-versus-host disease, systemic sclerosis, or combinations thereof.

27. The method of claim 26, wherein the polypeptide is an elastin-like polypeptide (ELP) and the cysteine-enriched segment is at the C-terminus of the polypeptide.

28. The method of claim 27, wherein the cysteine-enriched segment comprises (Gly-Gly-Cys)ₙ, wherein n is 2 to 10.

29. The method of claim 26, wherein the -L-D group of the conjugate or a pharmaceutically acceptable salt thereof has a structure of

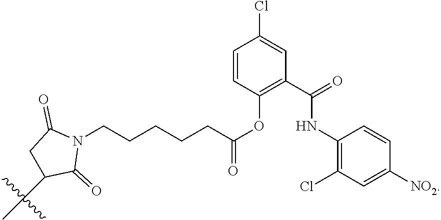 [SMILES: *C1CC(=O)N(CCCCCC(=O)Oc2ccc(Cl)cc2C(=O)Nc2ccc([N+](=O)[O-])cc2Cl)C1=O]

30. The method of claim 26, wherein p is 1, 2, 3, or 4.
